(19) United States
(12) Patent Application Publication
(10) Pub. No.: US 2011/0224300 A1
(43) Pub. Date: Sep. 15, 2011

(54) USE OF POTASSIUM 2-(a- HYDROXYPENTYL) BENZOATE IN THE MANUFACTURE OF MEDICAMENTS FOR PREVENTING AND/OR TREATING SENILE DEMENTIA

(75) Inventors: Xiaoliang Wang, Beijing (CN); Wanhong Zhao, Beijing (CN); Jinghua Yang, Beijing (CN); Ling Wang, Beijing (CN); Jiang Li, Beijing (CN); Shaofeng Xu, Beijing (CN); Nan Feng, Beijing (CN); Shiping Ma, Beijing (CN)

(73) Assignee: Institute of Mataria Medica, Chinese Academy of Me, Beijing (CN)

(21) Appl. No.: 13/003,045

(22) PCT Filed: Jul. 8, 2008
(86) PCT No.: PCT/CN2008/071588

§ 371 Date: Apr. 8, 2011

(51) Int. Cl. A61K 31/192; A61P 25/28

(52) U.S. Cl. 514/568

(57) ABSTRACT

The present invention discloses the use of potassium 2-(α-hydroxypentyl) benzoate in the manufacture of medicaments for preventing, relieving or treating senile dementia diseases or symptom, and for relieving oxidative stress injury in brain tissue, increasing neural function of choline, protecting neuron and/or raising the content of brain nerve growth factor. The present invention also discloses a pharmaceutical composition comprising potassium 2-(α-hydroxypentyl) benzoate in prophylactically or therapeutically effective dose, optionally, a pharmaceutically acceptable carrier and/or adjuvant.

### FIELD OF THE INVENTION

The invention concerns prevention or treatment of neurological diseases, in particular of aged dementia. It relates to the use of dl-PHPB for the prevention or treatment of aged dementia, or a symptom thereof. The aged dementia includes Alzheimer's Disease, vascular dementia, and a combination of both. It also relates to pharmaceutical compositions for prevention or treatment of aged dementia, which contain dl-PHPB and pharmaceutically acceptable excipients or vectors. Pharmaceutical compositions of dl-PHPB for the manufacture are chosen from solution, suspension, emulsion, pill, capsule, powder, control or continuous release preparation. The invention also relates to methods for the prevention or treatment of aged dementia, including providing an effective dosage of dl-PHPB or pharmaceutical compositions containing dl-PHPB to patients. The approach of providing dl-PHPB to patients can include external al, oral, local, intracutaneously, intramuscular, peritoneal, subcutaneous, intranasal, and so on.

### BACKGROUND OF THE INVENTION

Aged dementia includes Alzheimer's Disease (AD), vascular dementia (VD), and a combination of both. Aged dementia is a neurodegenerative disorder that is characterized by a progressive cognitive impairment and memory damage. This disease accounts for most dementias in old people, in particular aged above 60. In this disease the ability to remember, think, understand, communicate, and control behavior progressively declines because brain tissue degenerates. Many neurons in these brain regions contain large neurofibrillary tangles together with amyloid beta depositions. In China, the prevalence rate of aged dementia accounts for 4% in those above 65 years old. In the world, there are about fifty million patients having aged dementia. VD is a neurodegenerative disorder that is characterized by a cerebrovascular disease. In Europe and America, patients of VD account for 10-20% of aged dementia. In Asia, the incidents of VD are higher, such as Japan and China. China is entering the era of aging, with patients of aged dementia increasing year by year. Long term cerebral-ischemia is the main cause to form VD. Patients of aged dementia suffer themselves, and also impose a great burden for their family and society. There is no effective drug for treatment of aged dementia at present. Therefore, it is important to identify and develop effective drugs to control or treat progressive of AD and VD.

Potassium 2-(1-Hydroxypentyl)-benzoate (dl-PHPB), derived from 3-n-butylphthalide (NBP), is a newly synthesized compound that is under development as a therapeutic drug for cerebral ischemia. dl-PHPB was provided by the Department of Synthetic Pharmaceutical Chemistry of Chinese Academy of Medical Sciences with a purity of 99.9%. dl-PHPB is characterized by X-crystal diffraction, NMR, MS, infrared spectra, and HPLC-UV. The chemical structure of this compound is shown in FIG. 1. The preparation of dl-PHPB was described in PCT/CN02/1382682, entitled “novel salts of 2-(α-hydroxypentyl) benzoic acid, the methods for preparation and the application of these salts”.

There has been no report of using dl-PHPB for prevention or treatment of aged dementia thus far.

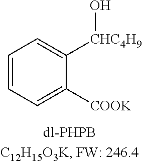

### Content of the Invention

In one aspect, the invention relates to the use of dl-PHPB for the prevention, amelioration or treatment of aged dementia, or a symptom thereof. It also relates to pharmaceutical compositions for prevention or treatment of aged dementia, which contain dl-PHPB and a pharmaceutically acceptable excipient or vector.

As used herein, aged dementia includes Alzheimer's Disease (AD), vascular dementia (VD), and conditions having a combination of both AD and VD. The symptoms of aged dementia can include memory impairment, impaired cognition, impaired thought process and impaired spatial orientation.

In this invention, the prevention is indicated to amelioration of the symptoms, including memory impairment, impaired recognition, impaired thought process and impaired spatial orientation, before the occurrence of the aged dementia, especially at its early stage. The therapy is indicated to the clinically significant improvement of the clinical symptoms, including memory impairment, impaired recognition, impaired thought process and impaired spatial orientation, during the progression of the aged dementia or after the diagnosis of Alzheimer's.

In another aspect, the invention relates to applications of dl-PHPB in alleviating oxidative stress damage, function of cholinergic neurons, protecting neuron and enhancing brain-derived neurotrophic factor (BDNF) in brain. The alleviated oxidative stress damage in the invention by dl-PHPB is indicated to decrease lipid peroxidation (Malondialdehyde, MDA)), and restore the balance of oxidation. The improved neurons function in the invention by dl-PHPB is indicated to enhance the activity of choline acetyltransferase (ChAT) and decrease the activity of acetylcholinesterase.

In the invention, optimistically chosen mammalian is human.

The aged dementia is a neurodegenerative disorder that is characterized by cognition defects, in particular short-term memory, progressively declined space orientation. Aged dementia can be induced by different causes, such as VD, neurofibrillary tangles together with amyloid beta depositions, and other age-related factors. The invention study adopted three publicly recognized dementia models: bilateral cervical carotid artery occlusion to simulate clinical VD syndromes, cognitive impairment induced by intracerebroventricular infusion of amyloid-beta (25-35) peptide in rats used to simulate clinical AD syndromes, and accelerated aging mice model (SAMP8) to simulate aging. dl-PHPB improved the learning and memory abilities of rats and mice in the Morris water maze test, the Y type water maze test and step down test. The results showed that dl-PHPB significantly alleviated cognitive impairment in bilateral cervical carotid artery occlusion (2-VO) model, intra-cerebroventricular infusion of amyloid-beta (25-35) peptide model, and accelerated aging mice model (SAMP8).

In summary, dl-PHPB significantly alleviated cognitive impairment in the three aged dementia models. Studies also showed that dl-PHPB significantly reversed the abnormalities of brain tissue induced by long-term cerebral-ischemia, inhibited the activation of astrocytes, and consequently protected neurons; and reduction of level of MDA, inhibition of oxidative stress damage; enhancement of the ChAT activity, increased level of acetylcholine and the improved learning and memory abilities; increased the level of BNDF after long-term cerebral-ischemia. Therefore, dl-PHPB might be a potential neuroprectant for the prevention or the treatment of the aged dementia.

The present invention is based on the findings that, in the long-term cerebral-ischemia model, the short-term memory and space orientation had been improved by dl-PHPB. VD is a neurodegenerative disease induced by cerebrovascular disorders, following cerebral artery occlusion, or low infusion, and lacunal cerebral stroke. The reduction of blood stream in brain relates to the aged dementia. Long-term cerebral-ischemia is contributed to the decreased utilization of oxygen, glucose and other necessary metabolites, consequently inducing oxidative stress damage, reduction of mitochondrion function and neuron biosynthesis, inhibition of transmission of synapse, and formation of neurodegenerative changes. The clinical VD syndromes include cognition defects, in particular short-term memory impairment, progressively declined space orientation.

The Morris water maze test is a typical experiment to evaluate short-term memory and space orientation abilities of the experimental animals. In the invention, the effect of dl-PHPB for improving short-term memory and space orientation was evaluated in bilateral cervical carotid artery occlusion (2-VO) model of rats. The results indicated that dl-PHPB significantly improved short-term memory and space orientation of the rats in 2-VO model. In one month after bilateral cervical carotid artery occlusion, the rats were examined by the escape latencies of place navigation test and the times of crossing the exact position of the former platform of probe trial test in Morris water maze test, in which spatial learning and memory were assessed.

In place navigation test, the escape latencies, searching strategy, and the swimming speed were investigated in Morris water maze test. The results indicated that dl-PHPB significantly improved learning and memory abilities of rats after bilateral cervical carotid artery occlusion. The effect of decreasing the escape latencies in 2-VO rat model, showed that dl-PHPB significantly improved short-term memory and space orientation of 2-VO rat model. The results of searching strategy showed that rats treated with dl-PHPB significantly increased the times of straightaway and tendency strategy, which indicated that dl-PHPB significantly increased space orientation abilities of 2-VO rat model. The speed of rats treated with dl-PHPB was not significantly different from the rats treated with saline, indicating the method for detecting effect of dl-PHPB on the improvement of the memory and learning abilities was not influenced by the physical strength of rats. The experiment results indicated dl-PHPB could prevent and treat aged dementia, in particular improve spatial learning and short-term memory capability of vascular dementia disease.

After place navigation testing, the platform of probe trial test were investigated in Morris water maze test, in which the retain time of target quadrant, and time of first crossing the platform location was record when the platform was removed to detect the memory capability of rats for the platform. The results showed that the rats treated with dl-PHPB significantly increased the retain time of target quadrant, and decreased the time of first crossing the platform location, compared to the mice treated with saline. The results indicate that dl-PHPB could improve spatial learning ability of 2-VO rat model. The experiment results indicated dl-PHPB could prevent and treat aged dementia, in particular improve spatial learning and memory capability of vascular dementia disease.

For clearing the mechanism of dl-PHPB for prevention and treatment of vascular dementia disease, the indices related to oxidant stresses damage and cholinergic system were detected, in particular the activity of superoxide dismutasen (SOD), level of MDA and activity of ChAT of the brain tissue of rats treated with dl-PHPB.

SOD is a major antioxidation enzyme in vivo, with radical scavenging and anti-oxidant damage functions. MDA is a major product of super oxidation. The activity of brain SOD reflected the capacity of anti-oxidation, whereas level of MDA reflected state of peroxidation. In one month after the onset of bilateral cervical carotid artery occlusion, the activity of SOD and level of MDA was significantly increased in cortex, compared to the rats treated with saline. The results indicated that dl-PHPB could alleviate the disordered anti-oxidation function in brain, decrease production of lipid peroxidation, restore the balance of oxidation. dl-PHPB might be used for the applications in antioxidant and free radical scavenger in brain tissue.

Acetylcholine is a major neurotransmitter, inducing signal transduction of cholinergic neurons and related to learning and memory. ChAT is an acetylcholine synthesis enzyme, the activity of ChAT reflected the function of cholinergic neurons. In one month after the onset of bilateral cervical carotid artery occlusion, the activity of ChAT significantly decreased in hippocampus. The rats treated with dl-PHPB for 21 days, significantly increased the activity of ChAT, and improved the function of cholinergic neurons. The results indicated that dl-PHPB might be used for application in enhancing function of cholinergic neurons.

The behavior in rats has been changed by bilateral cervical carotid artery occlusion, followed by the activation of glia cell and changes of white matter and grey matter. Moreover, for further clearing the mechanism of dl-PHPB on prevention and treatment of aged dementia diseases, pathology of rats treated with dl-PHPB was investigated by pathological and immunohistochemistrical approaches, including HE and Klüver-Barrera staining, detection of glial fibrillary acidic protein (GFAP) and BDNF. The results showed that the disorder of neurons in CA1 and CA3 sub region was significantly improved in rats treated by dl-PHPB. The result of experiments indicated dl-PHPB could protect and treat the damage of cortex and hippocampus induced by bilateral cervical carotid artery occlusion.

K-B staining could reflect the completeness of myelination of neurons and pathological changes of nerve fibers. The vacuole formation of corpus callosum and disturbance of optic tracts in rats treated by dl-PHPB was significantly improved, compared to the rats treated with saline. The result of experiments indicated dl-PHPB could protect the damage of corpus callosum and optic tracts by bilateral cervical carotid artery occlusion.

The effect of GFAP in glia cells was detected in rat treated with dl-PHPB by bilateral cervical carotid artery occlusion. Disturbances of brain tissue may be the basic of spatial learning and memory capability insulted by bilateral cervical carotid artery occlusion. The earliest phase of damage in brain tissue is white matter, following increase of astrocytes and activation of microglia. Glial fibrillary acidic protein (GFAP) is an intermediate filament (IF) protein that is thought to be specific for astrocytes. In the invention, cortex, corpus callosum, optic tracts, and hippocampus subregion were chosen to evaluate the effect of dl-PHPB. In cortex, the expression of GFAP in rats treated with saline was increased, compared with control group. The expression of GFAP in rats treated by dl-PHPB for 21 days was significantly decreased, compared to the rats treated with saline. In hippocampus, the expression of GFAP in rats treated with saline was significantly increased, compared with control group. The expression of GFAP in rats treated by dl-PHPB for 21 days was also significantly decreased, compared to the rats treated with saline. In corpus callosum, the expression of GFAP in rats treated with saline was not significantly different from the control rats. The expression of GFAP in rats treated by dl-PHPB for 21 days was decreased, compared to the rats treated with saline. In optic tracts, the expression of GFAP in rats treated with saline increased, compared with control group. The expression of GFAP in rats treated by dl-PHPB for 21 days was significantly decreased, compared to the rats treated with saline. In the above-mentioned experiment, dl-PHPB could significantly improve the damage of brain tissue by bilateral cervical carotid artery occlusion, decrease the activity of astrocytes, particularly in hippocampus, optic tracts, and cortex. The results of HE and K-B stain, expression of GFAP indicated dl-PHPB could be used to protect neuron.

In the invention, the effect of expression for BDNF in brain tissue was detected in rat treated with dl-PHPB by bilateral cervical carotid artery occlusion. BDNF acts on certain neurons of the central nervous system and the peripheral nervous system, helping to support the survival of existing neurons and encourage the growth and differentiation of new neurons and synapses. In the brain, it is active in the hippocampus, cortex, and basal forebrain—areas vital to learning, memory, and higher thinking BDNF itself is important for long-term memory. The expression of BDNF was increased in the earliest of cerebral-ischemia, and decreased at post-ischemia for 24 h. According to the results of immunohistochemistry, the expression of BDNF in cortex or hippocampus of vehicle group was significantly decreased, compared to control group. The expression of BDNF in cortex or hippocampus of rats treated by dl-PHPB for 21 days was significantly increased, compared to the rats treated with saline. The results showed dl-PHPB could increase the expression of BDNF in brain tissue of rats after bilateral cervical carotid artery occlusion. The results of expression of BDNF indicated dl-PHPB could be used to protect neuron.

In the invention, aged dementia rat model was induced by beta amyloid. dl-PHPB could improved the memory and space-learning abilities of rats treated with beta amyloid (25-35).

AD is a major cause to form neurodegenerative disorder that is characterized by a cognition defects. The pathological changes include neurofibrillary tangles together with amyloid beta depositions, and other age-related changes.

Amyloid beta (Aβ or Abeta) is a peptide of 39-43 amino acids that appears to be the main component of amyloid plaques in the brains of Alzheimer's disease patients. The aggregates of Aβ are related to damage of neuron, and cognition impairment. Aβ (25-35) is the major toxic peptide of Aβ, the toxicity of Aβ (25-35) is similar or more to Aβ (1-40) or Aβ (1-42). Research on laboratory suggest that symptom of rats treated by intracerebroventricular infusion (i.c.v.) of amyloid-beta (25-35) peptide is similar to clinical AD disease.

In the invention, the effect of dl-PHPB for improving short-term memory, space orientation ability was detected in rats treated by intra-cerebroventricular infusion (i.c.v.) of amyloid-beta (25-35) peptide. The results indicated that dl-PHPB significantly improved learning-memory, and space orientation ability of i.c.v. model in rats. In place navigation test, the dl-PHPB significantly decreased the escape latencies of i.c.v. model in rats in dose dependent manner. The results suggested that dl-PHPB significantly improved learning-memory, space orientation ability of i.c.v. rats model in dose dependent manner. The speed of swimming was no difference among each group for tasting period.

The results showed physical force of rats treated by intra-cerebroventricular infusion (i.c.v.) of amyloid-beta (25-35) peptide was no difference, compared to control group. The experiments results indicated dl-PHPB could prevent and treat aged dementia, in particular improved short-term memory capability of AD disease.

The platform of probe trial testing was investigated in Morris water maze test, in which the retain time of target quadrant, and time of first crossing the platform location was recorded when the platform was removed to detect the memory of rats for the platform. The results showed the rats treated with dl-PHPB significantly increased the retain time of target quadrant, and decreased the time of first crossing the platform location, compared to the mice treated with saline. The results indicated that dl-PHPB could improve spatial learning ability of i.c.v. rats model in dose dependent manner. The experiments results indicated dl-PHPB could prevent and treat aged dementia, in particular improve spatial learning and memory of AD disease.

For clearing the mechanism of dl-PHPB for prevention and treatment of AD disease, the indices related to oxidant stresses damage and cholinergic system were detected, in particular the activity of superoxide dismutasen (SOD), level of MDA and activity of ChAT in brain tissue of the rats treated with dl-PHPB.

At 14 days after intracerebroventricular infusion (i.c.v.) of amyloid-beta (25-35) peptide, the activity of SOD significantly increased, compared to the control rats. But the activity of SOD significantly decreased in cortex of rats treated with dl-PHPB for 14 days, compared to the rats treated with saline. The results indicated that dl-PHPB could decreased the activity of SOD in cortex of i.c.v. rats model in dose dependent manner.

At 14 days after intra-cerebroventricular infusion (i.c.v.) of amyloid-beta (25-35) peptide, the level of MDA significantly increased, compared to the control rats. But the activity of SOD significantly decreased in cortex of rats treated with dl-PHPB for 14 days, compared to the rats treated with saline. The results indicated that dl-PHPB could decrease the level of MDA in cortex of i.c.v. rats model in dose dependent manner. The results indicated that dl-PHPB could alleviate the disordered anti-oxidation activities in brain, decrease production of lipid peroxidation, restore the balance of oxidation. dl-PHPB might be used for antioxidant and free radical scavenger in brain tissue.

The activity of ChAT was not significantly changed in rats by intra-cerebroventricular infusion (i.c.v.) of amyloid-beta (25-35) peptide, compared to the control rats. But the activity of ChAT significantly increased in rats treated with dl-PHPB (39 mg/kg) for 14 days, compared to the rats treated with saline. The results indicated that dl-PHPB might be used to applicant for application to enhance function of cholinergic neurons.

In summary, dl-PHPB could significantly decrease the activity of SOD, and level of MDA in cortex of AD diseases at dose dependent manner. The results showed dl-PHPB could alleviate oxidative stress damage, decrease lipid peroxides (MDA), and restore the balance of oxidation in brain tissue. dl-PHPB might improve neurons function and enhance the activity of ChAT in AD disease.

In the invention, the effect of dl-PHPB for improving short-term memory and space orientation was detected in Samp8 mice. Senescence accelerated mouse (SAM) can be divided into two subtype, namely R subtype and P subtype. Clinical features of samP mice include deliplation, pachulosis, behavior disorder, survival period shortening, and so on. Clinical features of samR mice are similar to normal mice, following normal aging progress. The type of samP mice included 12 subtypes. Clinical features of samP8 mice majorly include memory defects by aging progress and subregion pathology of CNS (such as cortex and hippocampus). As reported, neurofibrillary tangles together with amyloid beta depositions and the changes of neurotransmitter were found in aging rats, including the decline of acetylcholine level, increase of opioid peptides, γ-aminobutyric acid and 5-hydroxytryptamine in cortex or hippocampus. Oxidative stress injury was found, following increase of brain lipid peroxide, oxidation-antioxidation system disturbance and dysfunction of mitochondria. In conclusion, SAMP8 may simulate senile dementia and its pathogenesis involves insufficient cortex mitochondrial function, decreased cholinergic nerve function, and oxidative stress.

In the invention, the effect of dl-PHPB for improving short-term memory and space orientation was detected in Samp8 mice by step down test. The results indicated that dl-PHPB could improve spatial learning and memory of SAMP8 mice. The experiment results indicated dl-PHPB could prevent and treat aged dementia, in particular improve spatial learning and memory capability of AD disease.

The step down test results showed the rats treated with dl-PHPB significantly decreased the foot shock time and increased the latent period, compared to the mice treated with saline. The experiment results indicated dl-PHPB could enhance the ability of active and passive avoidance response, improve the ability of learning and memory. The results showed that dl-PHPB could prevent and treat aged dementia, in particular improve spatial learning and memory capability of mixed dementia disease.

The maze step through test was used to detect the ability of short-term memory and space orientation of mice. The escape latency time and number of errors in encountering blind ends were used to reflect the ability of learning and memory in mice. In the invention, the mice treated with dl-PHPB had the shorter escape latency time and the litter number of errors in encountering blind ends, compared to the mice treated with saline. The results indicant that dl-PHPB could improve short-term memory and spatial learning capability of SAMP8 mice in dose dependent manner. The results showed that dl-PHPB could prevent and treat aged dementia, in particular improved spatial learning and memory capability of mixed dementia disease.

To clarify the mechanisms of dl-PHPB on prevention and treatment of aged dementia, in particular on improving mixed dementia disease, the biochemical indices related to aging, learning and memory, including activity of ChAT, AChE, SOD, and level of MDA, were detected in brain tissues by biochemical methods, The results showed that the dl-PHPB treatment on SAMP8 mice for 35 days, could significantly decrease the activity of SOD (p<0.05) and decrease level of MDA (p>0.05) in hippocampus, compared to the vehicle control. The results indicated that dl-PHPB could alleviate the brain anti-oxidation disorders, decrease production of lipid peroxidation, and restore the balance of oxidation.

Ach, one of the key neurotransmitters in the central nervous system, mediates the signaling pathway of cholinergic nerve and is closely involved in the learning and memory process. Choline acetyltransferase (ChAT) enzyme synthesizes the Ach and acetylcholinesterase (AChE) enzyme hydrolyzes the Ach, both of whose activities indirectly reflect the Ach content and function of cholinergic nerve in the brain.

After successive treatment up to 35 days, hippocampal ChAT activity of SAMP8 mice in the PHPB-treated groups has a statistically significant increase in comparison with control group and exhibited certain dose-dependency, which suggests PHPB might decrease AChE activity in the hippocampus of SAMP8 mice.

Therefore, PHPB raises the ChAT activity in the hippocampus of mixed-dementia patient and lessens the AChE activity in the hippocampus with certain tendency, which implies that PHPB might ameliorate the cholinergic function via increasing the ACh content in the hippocampus of mixed-dementia patient.

In summary, PHPB has preventive, ameliorative and therapeutic effects on vascular dementia. Its multiple actions are as followed: (1) PHPB significantly improves the recent memory and spatial location memory impairment; (2) PHPB notably lessens the SOD activity compensatorily increasing in the brain of vascular dementia patient and level of lipid peroxidation product MDA, which suggests that PHPB might inhibit the insult on the neuron induced by oxygen stress; (3) PHPB increases the ChAT activity in the brain of vascular dementia patient possibly inducing the higher ACh level in favor of improving learning and memory; (4) PHPB improves the pathologic change in the brain of vascular dementia patient including sparse white matter, vacuolization, an increase in glial cells and abnormal neuron morphology, and ameliorates the decrease of Brain-derived neurotrophic factors induced by brain ischemia.

Meanwhile, PHPB has preventive, ameliorative and therapeutic effects on presenile dementia. Its multiple actions are as followed: (1) PHPB significantly improves the learning and memory of presenile dementia patient; (2) PHPB notably lessens the SOD activity compensatorily increasing in the brain of presenile dementia patient and level of lipid peroxidation product MDA, which suggests that PHPB might inhibit the insult on the neuron induced by oxygen stress and protect the neurons; (3) PHPB increases the ChAT activity and improves cholinergic function, which helps ameliorating learning and memory impairments in the presenile dementia patient.

In addition, PHPB has preventive, ameliorative and therapeutic effects on mixed dementia. Its multiple actions are as followed: (1) PHPB significantly improves the recent memory and spatial location memory impairment; (2) PHPB notably lessens the SOD activity compensatorily increasing in the brain of mixed dementia patient and level of lipid peroxidation product MDA, which suggests that PHPB might protect neurons from the insult induced by oxygen stress; (3) PHPB increases the ChAT activity and possibly decreased AChE activity in the hippocampus, which suggests that it might improve learning and memory impairments in the mixed dementia probably via boosting cholinergic functions.

In brief, PHPB has therapeutic effects on the dementia, and ameliorative effects on cognitive decline associated with aging process, which mechanisms are involved in reduction of insults induced by oxygen stress in the brain, enhancement of functions in the cholinergic nerve, increase of brain-derived growth factor.

On the other hand, the invention refers to the drug composites used for the prevention, alleviation and treatment on the dementia-related signs, which includes the PHPB at prevention- or treatment-effective dose, as well as optional and pharmaceutical acceptable carriers and excipients. In the invention, drug composites could be prepared as the following formulations on the basis of administration route: solution, suspension, emulsion, pill, capsule, powder, controlled-release or sustained-release preparation.

The administration routes, in which PHPB composites prepared with known methods are administered in the invention, included but not limited to the following: parenteral, per os, focal, intracutaneous, intramusculary, intraperitoneally, subcutaneous, intranasal route.

PHPB in the invention could be prepared via the known methods.

Optional PHPB composite in the invention could be prepared with one or multiple pharmaceutical acceptable carriers and/or excipients via any routine method. Therefore, PHPB and its solvated forms could be specially prepared for inhalation, insufflations (via mouth or nose), per os, buccal, parenteral or rectum administration.

PHPB composites could also be taken in the form of solution, suspension, emulsion, pill, capsule, powder, controlled-release or sustained release formulation. These preparations contain PHPB at treatment-effective dose optimized for purified form and appropriate amount of carrier to provide patients proper administration options. The preparation should be consistent with administration route.

In the invention, the purified form of PHPB as stated refers to basically pure PHPB, especially with purity more than 80%, optimized pure PHPB with purity more than 85%, specially optimized PHPB with purity more than 90%, even more optimized PHPB with purity more than 98%. In summary, the purity of PHPB as stated above ranges from 95% to 99%, for instance.

### Parenteral Administration

PHPB composites could be prepared for parenteral administration via injection, for example, bolus infusion. The injection preparation lies in one ampoule as one unit formulation or multi-dose container with optionally additive preservatives. Parenteral formulation is placed into ampoules, disposable syringes or multi-dose containers made of glass or plastics et al. It could also be taken in the form of suspension, solution or emulsion containing lipophilic or hydrophilic carriers and excipients, such as deflocculant, stabilizer and/or disperser.

For example, the parenteral preparation is one kind of sterile injection or suspension with non-toxic, extra-parenterally acceptable diluents or solvents (for example, solution dissolved in, 3-butanediol). Acceptable carriers and available solvents includes water, Ringer's solution and iso-osmotic sodium chloride solution. In addition, sterile and non-volatile oil is routinely used as solvents or suspension medium. Given this, any gentle and non-volatile oil could be used including synthetic monoglyceride and diglyceride. Moreover, fatty acids such as oleic acid are also employed in the parenteral preparation.

In addition, PHPB composites could also be prepared into powder form, which needs reconstruction with proper vehicle such as pyrogen-free and sterile water before administration. For example, PHPB composites suitable for parenteral administration included sterile and iso-osmotic solution, which contained 0.1% to 90% PHPB by weight per volume. The content of PHPB in the solution is approximately 5% to 20%, optimized to approximately 5% to 17%, more optimized to approximately 8% to 14%, one more optimized to 10%. The solution or powder formulation might contain solubilizer, and local anesthetic such as lidocaine to ease the pain in the injection site. It's known for the other parenteral administration method in this domain, which is included in the scope of this invention.

### Oral Administration

PHPB composite could be prepared in the form of tablet or capsule via routine methods with pharmaceutical acceptable excipients such as adhesive, filler, lubricant and disintegrant.

### A. Adhesive

The adhesives are not only limited to maize starch, also including potato starch or other starch, gelatin, natural and synthetic gum such as arabic gum, algin, alginic acid, other alginate, powdered tragacanth, guar gum, cellulose and its derivatives (such as ethyl cellulose, cellulose acetate, calcium carboxymethylcellulose, sodium carboxymethyl cellulose), polyvinylpyrrolidone, methylcellulose, pregelatinized starch, cellulose hydroxypropyl methyl (such as, Nos. 2208, 2906, 2910), microcrystalline cellulose and its mixture. Proper formats of microcrystalline cellulose, for instance, included the materials sold as AVICEL-PH-101, AVICEL-PH-103 and AVICEL-PH-105 (from FMC Corporation, American Viscose Division, Avicel Sales, Marcus Hook, Pennsylvania, USA). An example of a suitable adhesive was the mixture of microcrystalline cellulose and sodium carboxymethyl cellulose sold as AVICEL RC-581 from FMC corporation.

### B. Filler

The fillers include talc, lactose, microcrystalline cellulose, powdered cellulose, dextrates, kaolin, mannitol, silicic acid, sorbitol, starch, pregelatinized starch and its mixture.

### C. Lubricant

The lubricants include calcium stearate, magnesium stearate, mineral oil, light mineral oil, glycerine, glucitol, mannitol, polyethylene glycol, other ethanediol, stearine, sodium lauryl sulfate, talc, hydrogenated vegetable oil (such as peanut oil, cotton oil, sunflower oil, sesame oil, olive oil, maize oil and bean oil), zinc stearate, aethylis oleas, Laurate ethyl, agar and its mixture. Other lubricants included, for example, solid silicone (AEROSIL 200, Baltimore, Md., USA, W.R. Grace Co.), condensation aerosol of synthetic silica (Deaussa Co. of Plano, Tex., USA), CAB-O-SIL (a kind of pyrogenic silica product, sold by Cabot Co., in Boston, Mass., USA) and its mixture.

### D. Disintegrant

The disintegrants include agar-agar, alginic acid, calcium carbonate, microcrystalline cellulose, croscarmellose sodium, crospovidone, polacrilin potassium, sodium starch glycolate, potato or cassava starch, other starch, pregelatinized starch, clay soil, other algin, other cellulose, gum and its mixture.

Optional coating method in the art could be employed for tablets or capsules. If adhesives and/or fillers are used in the PHPB composite, they are generally up to 50% to 99% by weight of the compound. On one hand, about 0.5% to 15% disintegrant by weight, especially about 1% to 15% disintegrant, could combine with PHPB. Lubricant is optional and its content is no more than 1% PHPB by weight. The methods about the preparation of solid oral formulation and pharmaceutical acceptable additives are described in the Marshall, Solid Oral Dosage Forms, Modern Pharmaceutics (Banker and Rhodes, Eds.), 7: 359-427 (1979). Other less typical formulations are well known in the art.

The formulation of solution, syrup or suspension could be employed for oral liquid preparation. Or, liquid preparation could be in the form of dried product, and be reconstructed via water or suitable carrier before use. These liquid preparations are prepared with routine methods via pharmaceutical acceptable additives such as deflocculant (for instance, sorbitol syrup, cellulose derivatives or hydrogenated edible fat), emulsifier (for example, lecithin or acacia gum), hydrophobic carrier (for instance, apricot oil, oleaginous ester, alcohol, or fractionated vegetable oil), and/or preservatives (for example, methyl p-hydroxybenzoate, propyl p-hydroxybenzoate or sobic acid). Optional buffer salt, flavoring agent, colorant, aromatics and sweetening agent could be added into these formations. Oral formation might also be prepared into controlled-drug-release dosage form. Preferably, Oral dosage form contain from 10% to 95% compound. In addition, in the invention PHPB composite could also be prepared into buccal tablet or lozenge. Other PHPB oral administration routes are known to the skilled in the art and included within the invention.

### Controlled-Release Administration

Controlled (sustained)-release formation is designed to prolong the action time and decrease the administration frequency of PHPB. This kind of preparation could also influence the onset time or other properties such as compound level in the blood, thereby affecting the emerging of adverse effects.

The controlled-release formation is designed to initially release certain PHPB attaining the therapeutically needed efficacy, then gradually and consecutively release additional PHPB to maintaining therapeutic level in long course. In order to keep approximately constant compound level, PHPB is released from the preparation at certain speed to replace the PHPB metabolized and/or secreted in the body. Many induced factors stimulates the controlled release of PHPB, such as change of pH, change of temperature, enzyme, water or other physiological conditions or molecules.

Controlled-release system, such as delivery pump, could apply the compound in similar way of insulin or chemotherapy agent delivered to target organ or tumor. In the system, PHPB usually combines with bio-degradable, bio-compatible polymer implants, which is characterized by PHPB release at selected site in the control of time. Examples of polymeric materials include polyanhydride, polyorthoesters, polyglycolicacid, polylacticacid, polyethylenevinylacetate and its copolymers and combinations. In addition, the controlled-release system is placed in the vicinity of therapeutic goal, resulting to only a fraction of whole-body dose needed.

In the invention, PHPB could be applied via other controlled-release methods or drug delivery system known to the skilled in the art, which includes such as hydroxypropyl methylcellulose, other polymermatrix, gel, permeable membrane, infiltration systems, multi-layer coating, particles, liposomes, microspheres, etc., or any combination of the above, with different mixing ratio to provide needed release spectrum. Other controlled-release methods of PHPB is known to the skilled in the art and included within the invention.

### Inhalation Administration

PHPB could also be applied by inhalation administration via different devices conveniently delivering to patients' lung. For example, metered-dose inhaler (“MDI”) contained suitable Low boiling point propellant in the tank, such as dichlorodifluoromethane, trichlorofluoromethane, dichlorotetrafluoroethane, carbon dioxide or other suitable gas, which directly deliver the compound to lung. MDI devices could be obtained from many suppliers, such as 3M Corporation, Aventis, Boehringer Ingleheim, Forest Laboratories, Glaxo-Wellcome, Schering Plough and Vectura.

In addition, dry powder inhaler (DPI) could also be employed to apply the compound. The DPI apparatus generally uses one mechanism, for example, the outbreak of the gas brings about cloud-form dry powder resulting in the inhalation by patients. The DPI apparatus is known in the art and available from many suppliers, such as Fisons, Glaxo-Wellcome, Inhale Therapeutic Systems, ML Laboratories, Qdose and Vectura. Multi-dose DPI (“MDDPI”) system is its popular variable form permitting more than one therapeutic dose application. The MDDPI apparatus is available from many companies, such as AstraZeneca, GlaxoWellcome, IVAX, Schering Plough, SkyePharma and Vectura. For example, gelatin capsules and cartridges referring to inhaler and insufflators could be prepared into power composite containing PHPB and power matrixes suitable for this system, such as lactose and starch.

Another kind of apparatus referred to the application of compounds to the lung, such as liquid spray device provided by Aradigm Corporation. Liquid compound is atomized via a tiny nozzle in the liquid spray system, which is directly inhaled into the lung. For example, atomizer device could be employed for the application of compounds to the lung. Using ultrasonic energy, liquid compounds are transformed into the aerosol in the atomizer, which consists of small particles prone to the inhalation. Examples of atomizers, include the devices provided by Sheffield/Systemic Pulmonary Delivery Ltd, Aventis and Batelle Pulmonary Therapeutics.

In another embodiment, electro-hydrodynamic (EHD) aerosol device is used for the application of compounds to the lung. Liquid solution or suspension is atomized via energy in the EHD aerosol device. When the compound is applied to the lung through EHD aerosol device, the electrochemical properties on the compound preparation is the optimized important parameters. The optimization is routinely carried on by the skilled in the art. Other lung delivery methods on PHPB are well known to the skilled in the art, and includes within the invention.

PHPB preparation suitable for the atomizer, liquid spray device and EHD aerosol device, generally consists of PHPB and pharmaceutically acceptable carriers. In a representative embodiment, the pharmaceutically acceptable carrier is a kind of fluid, such as alcohol, water, polyethylene glycol or perfluorocarbon. Optionally, the aerosol properties on the compound solution or suspension could be changed through the addition another substance. For example, the substance might be a kind of fluid, such as alcohol, diol, polyethylene glycol or fatty acids. Other preparative methods on the liquid compound solution or suspension suitable for the aerosol devices, are well known to the skilled in this art.

### Reservoir Administration

PHPB could also be prepared into reservoir preparation. Such prolonged action preparation is administered via implantation (such as subcutaneous or intramuscular) or intramuscular injection. Therefore, the compound could combine with suitable polymeric or hydrophobic materials, such as emulsion in the acceptable oil or ion exchange resin, or slightly soluble derivatives like slightly soluble salts. Other reservoir administration methods on the PHPB are well known to the skilled in this art, and includes within the invention.

### Focal Administration

For the focal administration, PHPB could combine with the carriers so as to deliver effective dose. According to required activity, the effective doses range from 1.0 μM to 1.0 mM. In one aspect of the invention, focal administration of the compound composite could be applied to the skin. Carriers include, but not limited to the form of ointment, cream, gelatin, paste, foam, aerosol, suppository, pad or gel stick.

Focal preparation could comprise therapeutically effective compound in the ophthalmologically acceptable vehicles, such as buffed salt solution, mineral oil, vegetable oil like corn oil or peanut oil, vasoline, Miglyol 182, alcohol solution, liposome or liposome-like product. Any of these compounds could also comprise preservatives, antioxidants, antibiotics, immunosuppressant and other biologically or pharmaceutically effective agents with no harmful effects on the compound. Other focal administration methods on the PHPB are well known to the skilled in this art, and includes within the invention.

### Other Delivery Systems

Other delivery systems are well known to the skilled in the art, and could be used for the application of the compound in the invention. Moreover, these and other delivery systems could be combined or modified to optimize the PHPB administration in the invention.

The invention also refers to a method on the prevention, relief and/or treatment of the dementia disease or symptom, including therapeutically effective dose of PHPB or drug composite containing PHPB administered to patient in need. Administration routes include, but not limited to parenteral, per os, focal, intracutaneous, intramusculary, intraperitoneal, subcutaneous, intranasal route.

In the invention, said therapeutically effective dose of PHPB is optionally from 0.5 to 200 mg/kg body weight, optimized for 1-150 mg/kg body weight, more preferred is 2-100 mg/kg body weight, more preferred is 3-50 mg/kg body weight, more preferred is 4-35 mg/kg body weight, and more preferred is 5-20 mg/kg body weight any dose between.

The determination principles of the therapeutically effective dose of dl-PHPB.

In the invention, the term “the therapeutically effective dose” means that the subjects with the necessary therapeutically effective dose of PHPB were determined according to disease and extend of disease. For example, the dose of cure, prevent, inhibit or prevent or at least part of inhibit or prevent the target disease or condition.

The toxicity and efficacy of dl-PHPB was determined to LD50 (50% lethal dose groups) and the ED50 (50% effective dose groups) by the standard Pharmaceutical approach in cell cultures or experimental animals. Therapeutically index of dl-PHPB was ratio between toxicity and efficacy of a treatment dose, namely the ratio of LD50/ED50.

The data obtained from cell culture experiments and animal studies can be used in the preparation of humans and other mammals dose range used. dl-PHPB in the dose optimization with minimal toxicity or no toxicity, including ED50 of the concentration of circulating plasma or other body fluids.

The dose rang of dl-PHPB depend on the dosage form and route of administration. In the invention, the effective dose of dl-PHPB was estimated according to dose of animal experiments. The dose of dl-PHPB was designed to achieve IC50 of plasma concentration in animal models. Then, the effective dose of dl-PHPB could be more accurately determine according to the information in the humans and other mammals. The level of dl-PHPB in plasma could be measured by high performance liquid chromatography.

The effective dose of a single dosage dl-PHPB combination with pharmaceutical acceptable carriers was determined according to the host and the specific and different delivery modes. The field technicians should understand that the content of dl-PHPB in individual dose of each formulation does not require achieving the effective dose by itself, because it was easy to achieve the required dose by applying to multi-individual dose of each formulation. The dose of dl-PHPB chosen depend on formulations, disease, and the specific purpose to be decided by the field technicians

The dose programs of dl-PHPB used to treat diseases was chosen to whether apply delivery system of dl-PHPB, according to multi-factors: including the type of patient, age, weight, sex, diet, medical condition, route of administration, and pharmacological factors such as activity, efficacy, the characteristic of pharmacokinetics and the distribution of toxicology. Therefore, the actual dose program of dl-PHPB may be very different between the subjects and the subjects.

### Terms and Abbreviations

dl-PHPB, PHPB is 2-(α-hydroxy-pentyl) benzoic acid potassium salt
SOD is superoxide dismutase
MDA is the malondialdehyde,
ChAT is the choline acetyltransferase,
AChE is acetylcholinesterase,
ATPase is the triphosphate phosphohydrolase.

### FIGURE LEGENDS

FIG. 1. Effects of dl-PHPB on the escape latencies of permanent 2-VO rats in water maze performance. Values are mean±S.E.M. (N=17-20). ^{#}P<0.05, ^{# #}P<0.01 v.s. sham group; *P<0.05 v.s.vehicle group (LSD test).

FIG. 2. The typical swimming-tracking paths of permanent 2-VO rats in Morris water maze. A: marginal mode; B: random mode; C: tendency mode; D: linear mode.

FIG. 3. Effects of dl-PHPB on the time in the platform-quadrant (A) and the first crossing-platform time (B) of permanent 2-VO rats in Morris water maze after administering for 21 days. Values are mean±S.E.M. (N=17-20). *P<0.05, ***P<0.01 v.s.vehicle group (LSD test).

FIG. 4. Effects of dl-PHPB on the biochemical indexes of brain tissues from permanent 2-VO rats. A: activity of SOD in cortex; B: level of MDA in cortex; C: activity of ChAT in hippocampus. Values are mean±S.E.M. (N=5-8). # # #P<0.001 v.s. sham group; *P<0.05, **P<0.01,***P<0.001 v.s.vehicle group (LSD test).

FIG. 5. Effects of dl-PHPB on photomicrographs of hematoxylin and eosin staining of cerebral cortexes of permanent 2-VO rats after administering for 21 days. Magnification, 400×

FIG. 6. Effects of dl-PHPB on photomicrographs of hematoxylin and eosin staining of hippocampus CA1 region of permanent 2-VO rats after administering for 21 days. Magnification, 400×

FIG. 7. Effects of dl-PHPB on photomicrographs of hematoxylin and eosin staining of hippocampus CA3 region of permanent 2-VO rats after administering for 21 days. Magnification, 400×

FIG. 8. Effects of dl-PHPB on photomicrographs of klüver-barrera luxol fast blue staining of corpus callosums of permanent 2-VO rats after administering for 21 days. Magnification, 400×

FIG. 9. Effects of dl-PHPB on photomicrographs of klüver-barrera luxol fast blue staining of optic tracts of rats after administering for 21 days. Magnification, 400×

FIG. 10. Effects of dl-PHPB on photomicrographs of the immunohistochemical staining for GFAP in the hippocampus of permanent 2-VO rats after administering for 21 days. Magnification, 200×

FIG. 11. Effects of dl-PHPB on photomicrographs of the immunohistochemical staining for GFAP in the optic tracts of permanent 2-VO rats after administering for 21 days. Magnification, 200×

FIG. 12. Effects of dl-PHPB on active astrocytes in brain of permanent 2-VO rats after administering for 21 days. A: cortex, B: hippocampus, C: corpus callosum, D: optic tract. Values are mean±S.E.M. (N=4). P<0.01 v.s. sham group; *P<0.05, **P<0.01 v.s.vehicle group (LSD test).

FIG. 13. Effects of dl-PHPB on the area and density of BDNF in cortex of permanent 2-VO rats after administering for 21 days. Magnification, 200×

FIG. 14. Effects of dl-PHPB on photomicrographs of the immunohistochemical staining for distribution and content of GFAP in the hippocampal CA1 area of permanent 2-VO rats after administering for 21 days. Magnification, 200×. A: sham group; B: vehicle group; C: PHPB 39 mg/kg group.

FIG. 15. Effects of dl-PHPB on photomicrographs of the immunohistochemical staining for distribution and content of GFAP in the hippocampal CA2 area of permanent 2-VO rats after administering for 21 days. Magnification, 200×. A: sham group; B: vehicle group; C: PHPB 39 mg/kg group.

FIG. 16. Effects of dl-PHPB on photomicrographs of the immunohistochemical staining for distribution and content of GFAP in the hippocampal CA3 area of permanent 2-VO rats after administering for 21 days. Magnification, 200×. A: sham group; B: vehicle group; C: PHPB 39 mg/kg group.

FIG. 17. Effects of dl-PHPB on the content of BDNF in brain tissue of permanent 2-VO rats after administering for 21 days. Values are mean±S.E.M. (N=4). ^{#}P<0.05, v.s. sham group; *P<0.05, **P<0.01 v.s.vehicle group (LSD test).

FIG. 18. Effects of dl-PHPB on the escape latencies of Aβ(25-35)-induced dementia rats in Morris water maze performance after administration. Values are mean±S.E.M. (N=7-9). ^{#}P<0.05, v.s. sham group; *P<0.05, **P<0.01 v.s.vehicle group (LSD test).

FIG. 19. Effects of dl-PHPB on the time in the target-quadrant and the first crossing-platform time of Aβ(25-35)-induced dementia rats in Morris water maze performance after administration. A: dl-PHPB increased dose-dependently the time in the target-quadrant of dementia rats compared with the vehicle rats; B: dl-PHPB had a tendency of reducing the first crossing-platform time. Values are means±S.E.M. (n=7-9). #P<0.05 vs. sham group; *P<0.05 vs. vehicle group (LSD test).

FIG. 20. Effects of dl-PHPB on the biochemical indexes of Aβ(25-35)-induced dementia rats after administering for two weeks. A: cortex SOD activity; B: cortex MDA level; C: the cortex ChAT activity. Values are means±S.E.M. (n=7-9). ^{#})<0.05 vs. sham group; *P<0.05, **P<0.01 vs. vehicle group (Dunnett or LSD test).

FIG. 21. Effects of dl-PHPB on the shock number and latency of eleven-month-old SAMP8 in step down test after administering for 30 days. A: dl-PHPB reduced dose-dependently the shock number suffered by SAMP8; B: dl-PHPB increased dose-dependently the shock latency of SAMP8. Values are means±S.E.M. (n=11-14). *P<0.05, **P<0.01, ***P<0.001 vs. control group (Dunnett test).

FIG. 22. Effects of dl-PHPB on the numbers of SAMP8 entering non-exit and the latencies of SAMP8 finding the steps in water maze after administering for 32-35 days. A: number of SAMP8 entering non-exit; B: latency of SAMP8 finding the steps. Values are means±S.E.M. (n=11-14). *P<0.05, **P<0.01 vs. control group (Dunnett test).

FIG. 23. Effects of dl-PHPB on the SOD activity and MDA level of brain tissues from SAMP8. A: hippocampus SOD activity; B: hippocampus MDA level. Values are means±S.E.M. (n=11-14). ^{#})<0.05 vs. sham group; (LSD test).

FIG. 24. Effects of dl-PHPB on the ChAT and AChE activity of brain tissues from SAMP8. A: hippocampus ChAT activity; B: hippocampus AChE activity; C: cortex ATPase activity of Mitochondria. Values are means±S.E.M. (n=11-14). ^{#})<0.05 vs. sham group; (LSD test).

FIG. 25. The experiment design and schedule of example 1.

FIG. 26. The experiment design and schedule of example 2.

FIG. 27. The experiment design and schedule of example 3.

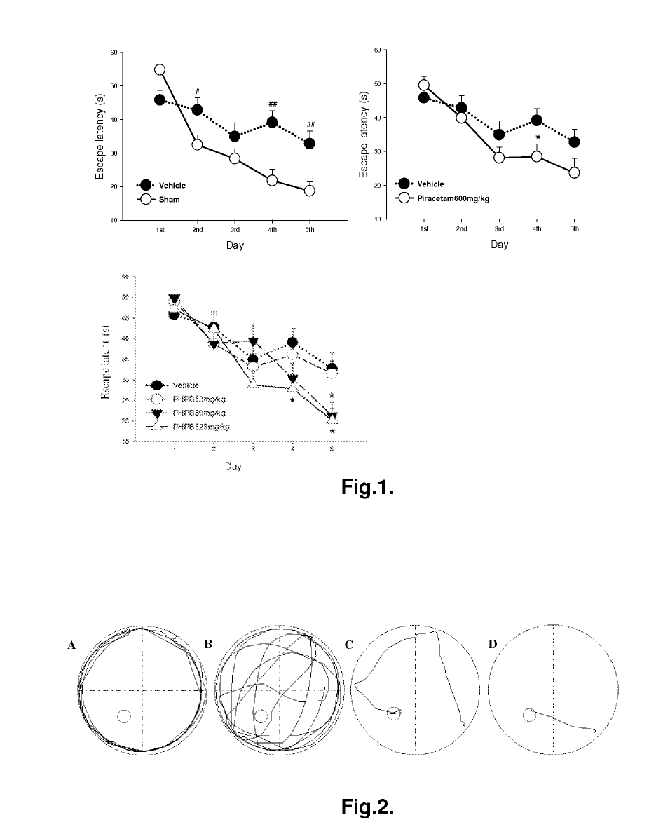

### THE SPECIFIC IMPLEMENTATIONS

The implementation does not limit the applications of the invention.

### Example 1

### The Study of Chronic Cerebral Ischemia in Rat: dl-PHPB Improves the Short-Term Memory and Spatial Learning Capability of Chronic Cerebral Ischemic Rat

### 1. Materials and Methods

dl-PHPB was offered by the department of medical synthetic chemistry of our institute with a purity of more than 98.5%. dl-PHPB was dissolved in distilled water. Piracetam tablets were purchased from Tianjin Jinshi Pharmaceutical limited company. The levels of SOD, ChAT, and MDA activities were determined with commercial colorimetric assay kits (Nanjing Jiancheng Bioengineering Institute, China). The content of protein in the supernatant was determined by Bradford method using BSA as the standard. Neutral red and fastness blue dyes were purchased from Sigma. Lithium carbonate was purchased from Beijing Chemical reagent limited company. Triton X-100 purchased from zhongshan goldenbridge biotechnology co., LTD. Antibody of GFAP purchased from Chemicon. Antibody of BDNF purchased from Santa Cruz Biotechnology co., LTD. Other reagents purchased from zhongshan goldenbridge biotechnology co., LTD.

### 2. Instruments

The water maze apparatus was designed by Institute of Material Medica, Chinese Academy of Medical Sciences. Enzyme mark instrument (MQX 200) was purchased by Bio Tek Instruments. Paraffin section machine (IR2135) purchased from German Leica co., LTD. Thermostatic freezing section machine (620-E) purchased from UK Shandon co., LTD. Automatic microphotography system (Nikon ECLIPSE 80i) purchased from Japanese Nikon Corporation.

### 3. Preparation of 2-VO Rat Model

Chronic cerebral hypoperfusion was proceeded by a permanent, bilateral occlusion of the common carotid arteries (2-VO) of adult rats. The rats were anesthetized with 10% trichloro-acetaldehyde, and temperature probe was inserted into the rectum, and a separate heating lamp was used to maintain rectal temperature at normothermic level. After a midline neck skin incision, the common carotid artery were exposed and ligated by a 5-0 nylon suture.

### 4. Treatment Groups and Drug Administration

Rats were randomly divided into six experimental groups (20 rats/each group): one sham-operated group, one vehicle control group, one piracetam-treated group (600 mg/kg), and three dl-PHPB-treated groups (13, 39, and 129 mg/kg). In 10 days after onset of 2-VO, drugs and vehicle were administered orally to rats for 21 days (one time/each day). The spatial learning and memory capability of rats were detected by Morris water maze in the 25-30 days after operation. The Morris water test was commenced at 40 min after drugs treated. The biochemical and pathology assays were conducted later (in 24 h) after behavioral tests. The experimental schedule is shown in FIG. 25.

### 5. Morris Water Maze

Subjects were tested in a Morris water maze. This test is based on a standardized assessment of spatial learning ability. The water maze apparatus consisted of a circular, stainless pool (120 cm in diameter, 60 cm in height). It was placed in a dimly lit, sound-proof test room. Multiple distant cues around the room (window, cabinets, furniture) were kept in the same location throughout the experiments. The water was filled to a depth of 40 cm at 25±1° C. and was made opaque by adding milk powder to prevent visualization of the platform. A transparent platform (10 cm diameter) was put 1.5 cm below the surface of the water. The tank was divided into four quadrants with the platform in a fixed position in one quadrant. The subjects were placed into the maze facing the pool wall and were allowed two trials per day, 60 s per trial, to find the hidden platform. If the subject found the platform within the 60 s, it was given a 10-s rest period on the platform between trials. If the pedestal was not located within the time allotted, the subject was placed on to the platform and allowed 10 s until the next trial. The escape latency (time to reach the platform) was used to assess acquisition of the water maze task. Sessions were repeated for five consecutive days. On the sixth day, the platform was removed and the rat was allowed to search for the platform for 60 s (probe test). The time in the platform quadrant and latency time to cross platform location were recorded to measure the spatial learning ability without the influence of chance encounters with the platform. The Morris water maze sessions were recorded with a video camera for offline analysis.

### 6. Biochemical Assays

The biochemical assays were conducted later (in 24 h) after behavioral testing. Eight rats in each group were anesthetized with ether and sacrificed. The brains were quickly removed and cleaned with ice-cold saline. Then the parietal cortex and hippocampus were isolated. For biochemical analysis, the tissues were weighed and homogenized in ice-cold saline with the proportion of 1:9 (w/v). Homogenization (IKA, Germany) was carried out for 2 min in an ice bath. After the homogenates were centrifuged at 2,000×g for 10 min at 4° C. (Sigma, Germany), the supernatant was used for analytical procedures. The levels of choline acetyltransferase (ChAT) SOD, and MDA activities were determined with commercial colorimetric assay kits (Nanjing Jiancheng Bioengineering Institute, China). The content of protein in the supernatant was determined by Bradford method using BSA as standard.

### 7. Histopathology and Immunohistochemistry

Hematoxylin-Eosin and Klüver-Barrera Staining

Four rats in each group were anesthetized with sodium pentobarbital (100 mg/kg, intraperitoneal injection), and perfused transcardially with cold saline, followed by 300 ml of 4% paraformaldehyde in 0.01 M phosphate buffer (pH 7.4). After these procedures, the brains were removed and stored in the 0.01 M phosphate buffer (pH 7.4) with 4% paraformaldehyde and 30% sucrose at 4° C. until fully equilibrated. Then they were postfixed at 4° C., dehydrated and embedded in paraffin blocks. Coronal sections of 8 μm were stained with hematoxylin-eosin and Klüver-Barrera.

### GFAP and BDNF Immunolabeling

The method for tissue fixation was described above. Serial coronal sections (40 μm) of the brains were cut through the dorsal hippocampus on a freezing microtome and collected in 6-well plates containing 0.01 M PBS. The detection of GFAP and BDNF immunoreactivity was performed using a conventional avidin-biotin-immunoperoxidease technique.

For quantitative analysis, one from every five samples in a continuous series of hippocampal or cortical tissue sections was taken, and was processed immunohistochemically. Thus, three slides were taken from each rat, and were read under an objective (×1.6) microscope. The total area of positively stained neurons was counted with Image-pro Plus 5.0 software.

### 8. Statistical Analysis

The results were expressed as mean±SEM. The data from training trail in the Morris water maze were analyzed by two-way analysis of variance (ANOVA) to detect the difference between groups and over time. The post hoc Dunnett's test was used to test the differences between two groups. Probe trial, biochemical assay, and immunohistochemistry assay were statically analyzed using one-way ANOVA followed by post hoc Dunnett's test. The results were considered to show a significant difference when the p value was less than 0.05.

### Results

1. dl-PHPB Significantly Alleviated Chronic Cerebral Hypoperfusion-Induced Impairments and Improved Spatial Learning and Spatial Working Memory in Rats.

Rats were given dl-PHPB daily for 21 days since the 10th day after operation. And they were tested in the Morris water maze in the 25-30 days after operation. The escape latency was used to reflect an aspect of cognition and spatial learning. Two-way ANOVA with repeated measures revealed a significant day effect on escape latency within groups, indicating that all group of rats improved their performance over the 5-day training period. Also, we found a significant treatment effect on escape latency.

Post hoc analysis confirmed the internal validity of the study. 2-VO rats showed a significant longer time to find the platform (escape latency) than sham-operated rats (p<0.01). dl-PHPB treated rats performed better on the fifth day (as indicated by statistical analysis), whereas sham operated rats performed better from the second day. The results suggested that chronic cerebral hypoperfusion successfully induced a learning defect. Second, we found that 13 mg/kg dl-PHPB-treated rats did not differ from the vehicle control animals. These results indicate that daily administration of 39 and 129 mg/kg dl-PHPB significantly rescued learning impairment caused by chronic cerebral hypoperfusion in escape latency in the water maze task (FIG. 2, Tab. 1 and 2).

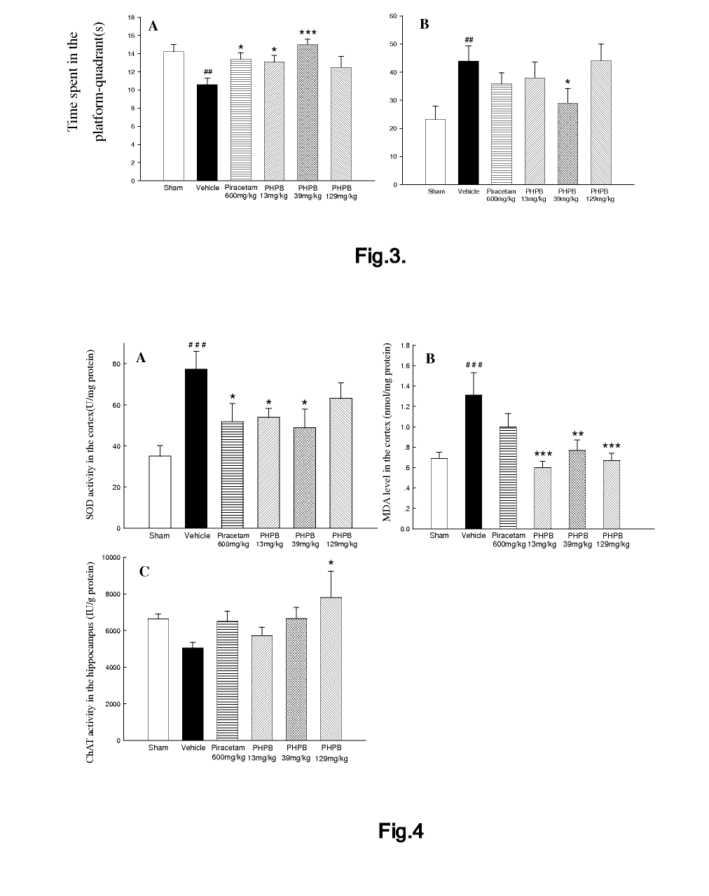

In probe trial day when the platform was moved, rats that have learned the location of the hidden platform are expected to spend the majority of the trial searching for the platform in the target quadrant. 2-VO rats spent less time in the platform quadrant (p<0.01) and took more time to cross the platform location (p<0.01) than sham-operated animals. dl-PHPB (13 and 39 mg/kg) markedly showed ameliorative effect compared to 2-VO animals. (FIG. 3 A-B)

These results suggested that 2-VO rats underwent spatial cognition impairments, and dl-PHPB attenuated the learning and memory damages in 2-VO rats.

TABLE 1
Effects of dl-PHPB on the navigation experiment in Morris water maze from permanent
BCCAO rats.

| Group | M | R | T | L | M | R | T | L | M | R | T | L | M | R | T | L | M | R | T | L |
|---|---|---|---|---|---|---|---|---|---|---|---|---|---|---|---|---|---|---|---|---|
| Sham | 7 | 19 | 14 | 0 | 2 | 6 | 26 | 6 | 0. | 6 | 27 | 7 | 0 | 4 | 24 | 12 | 0 | 6 | 17 | 17 |
| Vehicle | 10 | 10 | 15 | 1 | 8 | 11 | 14 | 3* | 5 | 6 | 22 | 3 | 2 | 12 | 16 | 6* | 1 | 7 | 22 | 6* |
| Piracetam | 10 | 15 | 14 | 1 | 3 | 11 | 17 | 8 | 1 | 6 | 23 | 10 | 1 | 7 | 23 | 10 | 1 | 8 | 12 | 19 |
| (600 mg/kg) | | | | | | | | | | | | | | | | | | | | |
| dl-PHPB | 11 | 13 | 11 | 1 | 3 | 11 | 16 | 6 | 0 | 10 | 21 | 5 | 0 | 9 | 20 | 7 | 0 | 5 | 26 | 4 |
| (13 mg/kg) | | | | | | | | | | | | | | | | | | | | |
| dl-PHPB | 7 | 15 | 12 | 2 | 3 | 9 | 18 | 4 | 1 | 13 | 17 | 3 | 0 | 7 | 18 | 9 | 0 | 4 | 13 | 17* |
| (39 mg/kg) | | | | | | | | | | | | | | | | | | | | |
| dl-PHPB | 4 | 16 | 10 | 4 | 2 | 12 | 19 | 1 | 0 | 7 | 21 | 6 | 1 | 5 | 20 | 7 | 0 | 2 | 23 | 9 |
| (129 mg/kg) | | | | | | | | | | | | | | | | | | | | |

The data expressed by search strategy times (N = 17-20),
*p < 0.01, v.s.sham-operation group;
M: marginal mode,
R: random mode;
T: tendency mode;
L: linear mode.

2. Effects of dl-PHPB on the SOD, ChAT Activity and MDA Level of Brain Tissues from Permanent BCCAO Rats.

SOD plays important roles in maintenance the balance of oxidation, antioxidant with radical scavenging action. However, MDA is a major peroxide. The activity of SOD and the level of MDA reflect the level of antioxidation in brain tissue. After behavioral testing, the rats were sacrificed and the activities of ChAT, SOD, and MDA in the cortex and hippocampus were measured. The results are shown in FIG. 4. In the cortex of 2-VO rats, the SOD activity (77.39±8.70 U/mg protein) was markedly higher than in the sham-operation group (35.03±5.20 U/mg protein, P<0.001), and MDA level was increased from 0.69±0.06 nmol/mg protein in sham-operation group to 1.31±0.22 nmol/mg protein in 2-VO rats (p<0.001). dl-PHPB at 13 and 39 mg/kg and piracetam at 600 mg/kg significantly alleviated the increase of SOD activity in 2-VO rats. Meanwhile, dl-PHPB treatment decreased MDA level at the dose of 13, 39, and 129 mg/kg in cortex of 2-VO rats. However, piracetam at 600 mg/kg did not show significant effects on MDA in cortex of 2-VO rats. Furthermore, no significant effects of dl-PHPB and piracetam on the SOD activity and MDA level were observed in the hippocampus of 2-VO rats.

Acetylcholine is a neurotransmitter in the central nervous system, mediated by cholinergic nerve signaling. Acetylcholine is closely related with learning and memory, and synthesized by ChAT. So, the activity of ChAT can indirectly reflect levels of acetylcholine and the status of cholinergic function. Among rats with permanent occlusion of bilateral common carotid arteries for 1 month, the ChAT activity was decreased significantly (a decrease of 24%) in the hippocampus of 2-VO rats, compared with sham operation group. After administration of 21 days, dl-PHPB 129 mg/kg significantly increased the activity of ChAT in hippocampus (P<0.05). However, dl-PHPB at 13, 39 mg/kg and piracetam at 600 mg/kg did not show significant effects on activity of ChAT in hippocampus of 2-VO rats (FIG. 4C). In the cortex, dl-PHPB and piracetam had no effect on ChAT activity (data not shown).

In conclusion, dl-PHPB can reduce SOD activity, and also decrease level of MDA in the cortex of 2-VO rats. The results indicated that dl-PHPB could improve disorders of the anti-oxidation in brain, decrease production of lipid peroxidation, and restore the balance of oxidation. In addition, dl-PHPB may improve ChAT activity and cholinergic function in 2-VO rats.

3. Effects of dl-PHPB on the Neuronal Morphology in 2-VO Rats

(1) HE Staining

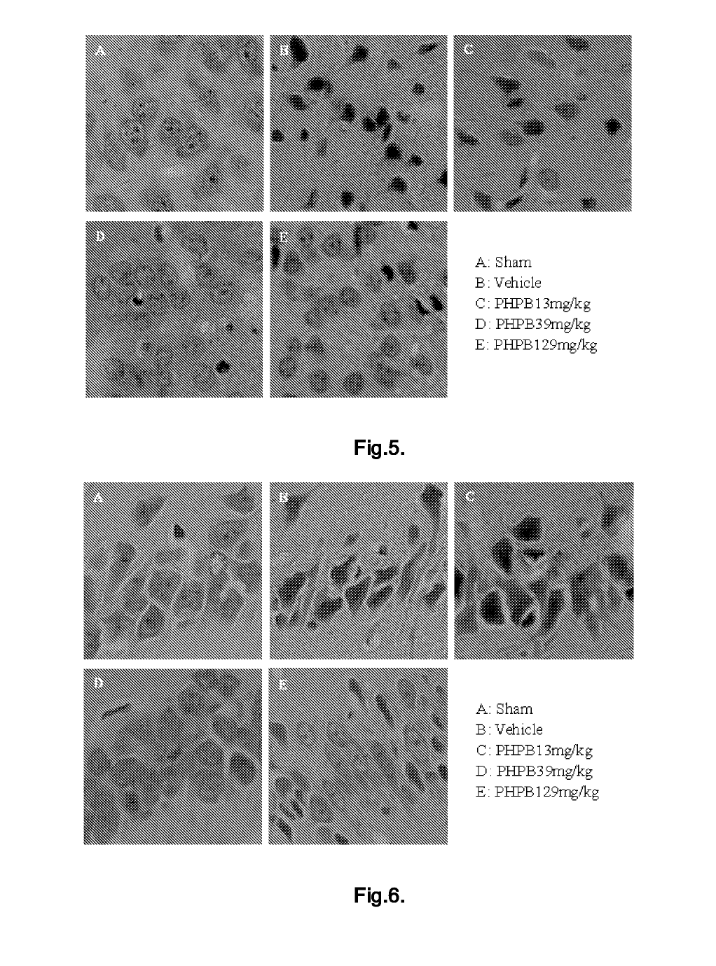

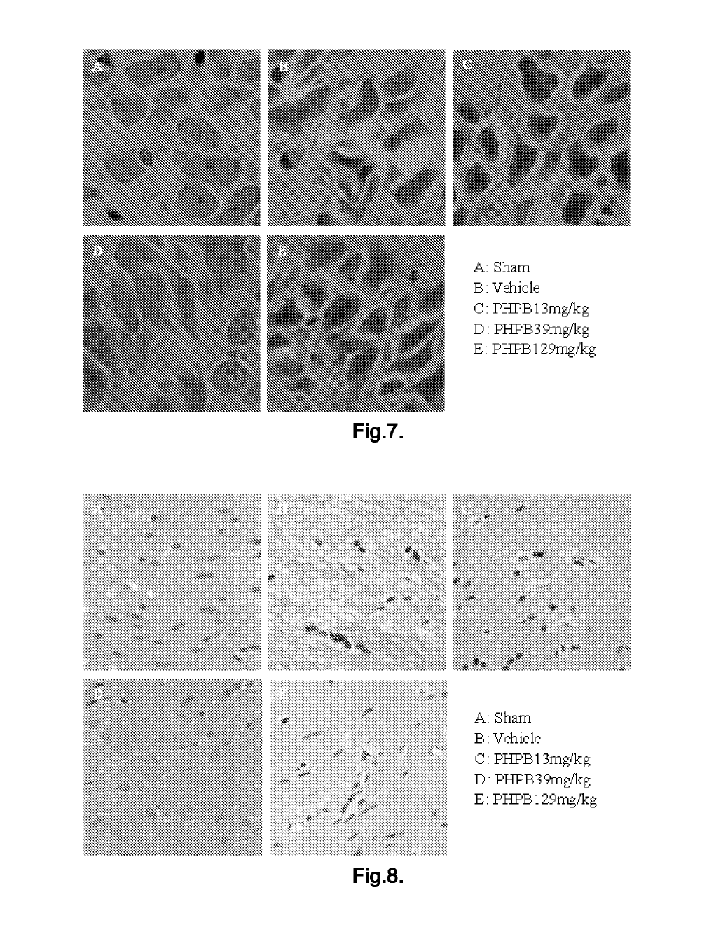

HE staining in the cytoplasm and nucleus in different colors, you can clearly see the general shape of the cells. Sham operation group in this study shows that cortical neurons shrink, deeply stained, nuclear is not clear, hippocampal CA1, CA3 areas also appear similar to the changes, but to a lesser extent, the hippocampus CA2 area were not affected. dl-PHPB 39 mg/kg could significantly improve the cortex, hippocampus area CA1 and CA3 neurons form. dl-PHPB 129 mg/kg could improve the cortex and hippocampus CA1 area neuronal morphologic abnormalities, and dl-PHPB 13 mg/kg only minor improve the abnormal shape of cortical neurons (FIG. 5-7). Above that, dl-PHPB could protect and care the damage of cortex and hippocampus neurons caused by chronic cerebral hypoperfusion.

(2) K-B Staining

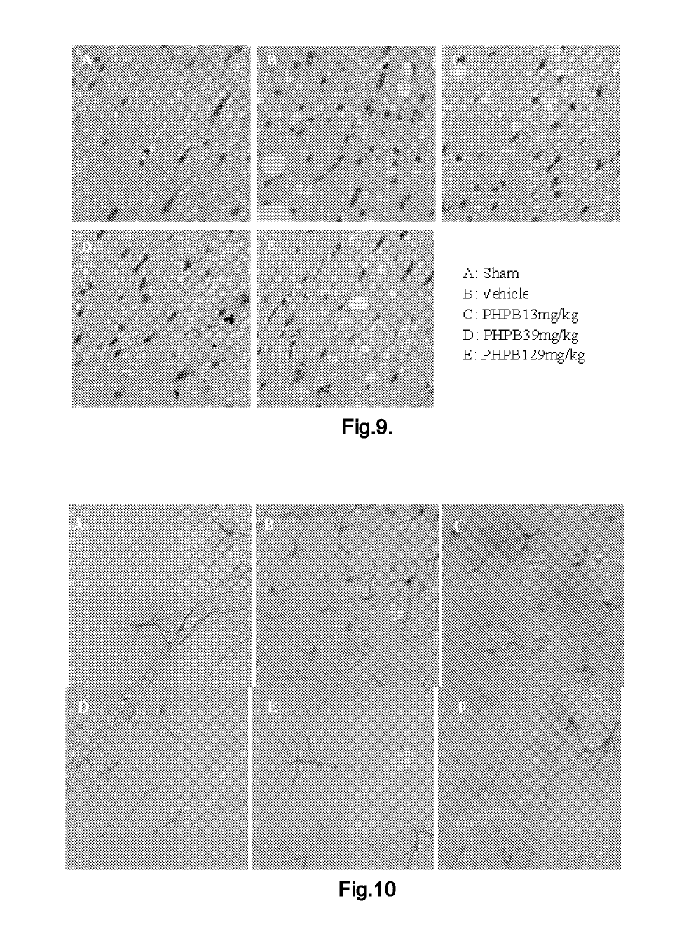

KB staining could reflect the morphological changes of neuronal myelin sheath, and the morphological changes of nerve fibers. It was found that the corpus callosum and optic tract showed clear vacuolization and nerve fiber disorders in sham operation group. dl-PHPB significantly improved the pathological damage of the corpus callosum, reduced vacuolization and restored nerve fiber arrangement, including dl-PHPB 39 mg/kg strongest, dl-PHPB 129 mg/kg followed, dl-PHPB 13 mg/kg the weakest (FIG. 8.) dl-PHPB also has a certain improvement in the pathological changes of the optic tract, which the strong role of dl-PHPB 39 mg/kg (FIG. 9). These results suggest that, dl-PHPB could significantly protect injury of the corpus callosum and optic tract in 2-VO rats.

4. Effects of dl-PHPB on the Expression of GFAP in Astrocytes of 2-VO Rats

Cerebral damage caused by hypoperfusion may be the basis for spatial learning and memory impairment, including earliest white matter damage, accompanied by an increase of astrocytes and microglia activation. Glial fibrillary acidic protein (GFAP) immunohistochemical staining can be used to label the activated astrocytes. 2-3 photos were taken from each selected slices in the same regions. The number of the GFAP positive astrocytes was calculated as the average value of the region. Four anatomical regions were selected to observe: the cortex, hippocampus, corpus callosum and optic tract.

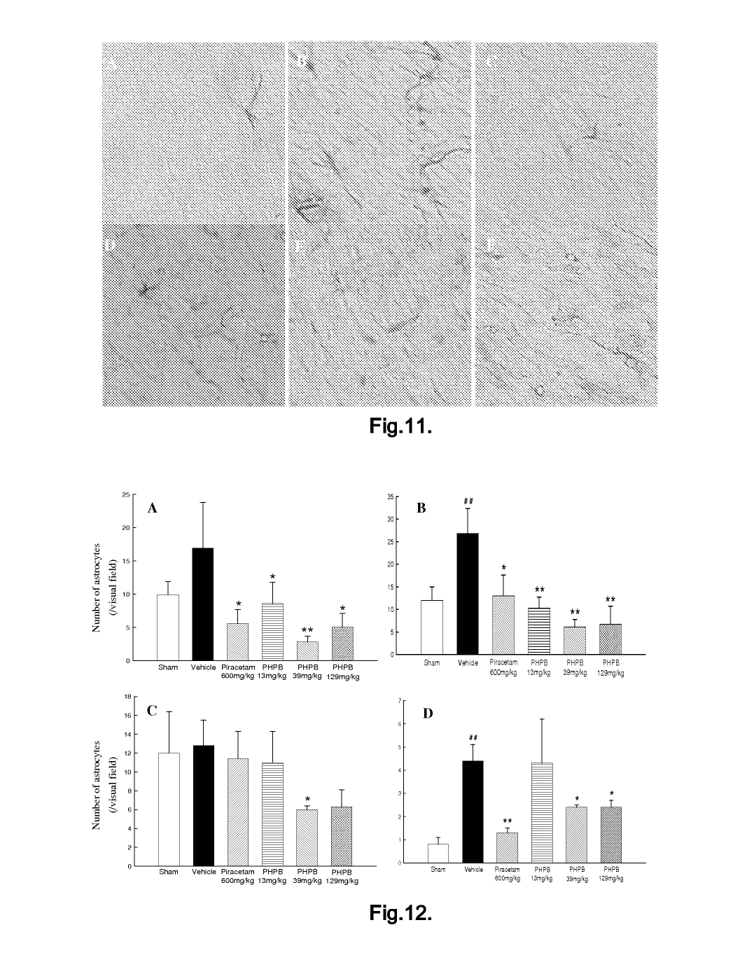

The results showed that the GFAP positive cells increased in the cortex of 2-VO rats (16.9±6.9), but not statistically significantly. While for dl-PHPB and Piracetam administration of 21 days, GFAP-positive cells were significantly less than in the vehicle group, in particular, at the dose of 39 mg/kg dl-PHPB (P<0.01) (FIG. 12A). In the hippocampus, GFAP positive cells significant increased in 2-VO rats (26.8±5.5), compared with sham operation group (12.0±3.0, P<0.01). While for dl-PHPB and Piracetam administration of 21 days, GFAP-positive cells were significantly less than in the vehicle group (P<0.05 or P<0.01), in particular, at the dose of 39 mg, 129 mg/kg of dl-PHPB with the most significant effect (P<0.01) (FIGS. 10 and 12B). In the corpus callosum, there are no significant differences between sham operation group and vehicle group, but the dl-PHPB 39 mg/kg significantly decreased level of GFAP positive cells (P<0.05) (FIG. 12C). In the optic tract, GFAP positive cells significantly increased in 2-VO rats (4.4±0.7), compared with sham operation group (0.8±0.3, P<0.01). While dl-PHPB 39,129 mg/kg and Piracetam after administration for 21 days, GFAP-positive cells were significantly less than the vehicle group (P<0.05 or P<0.01) (FIGS. 11 and 12D). As described above, dl-PHPB can significantly alleviate cerebral damage of 2-VO rats and reduce the number of activated astrocytes, particularly in the hippocampus, optic tract and cortex, with the strongest effect at the dose of 39 mg/kg, succeeded by 129 mg/kg and 13 mg/kg.

5. Effects of dl-PHPB on the Expression of BDNF in 2-VO Rats

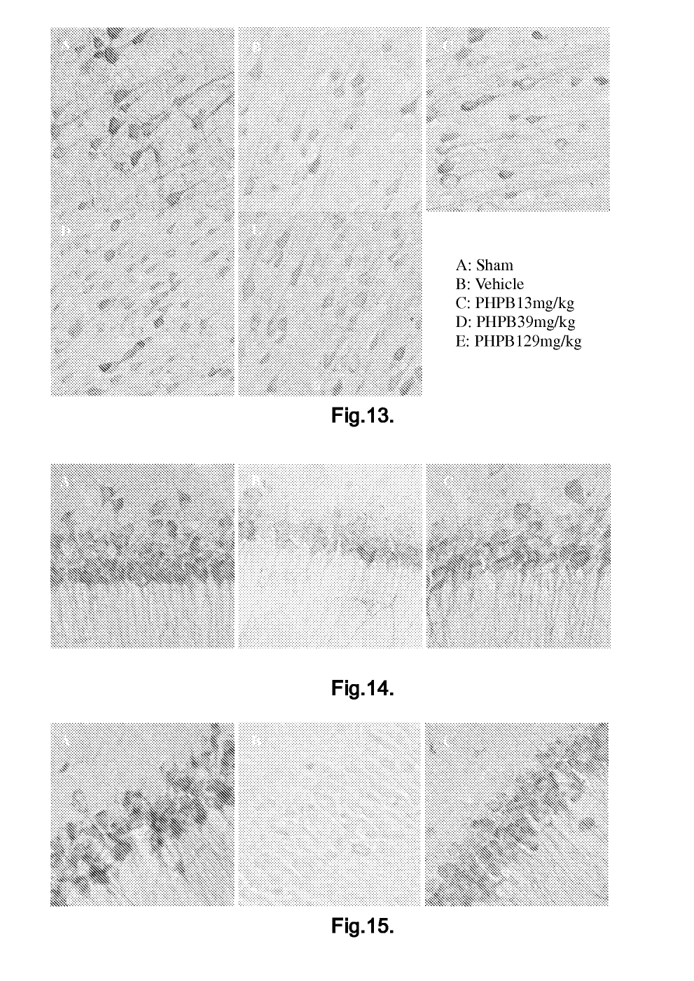

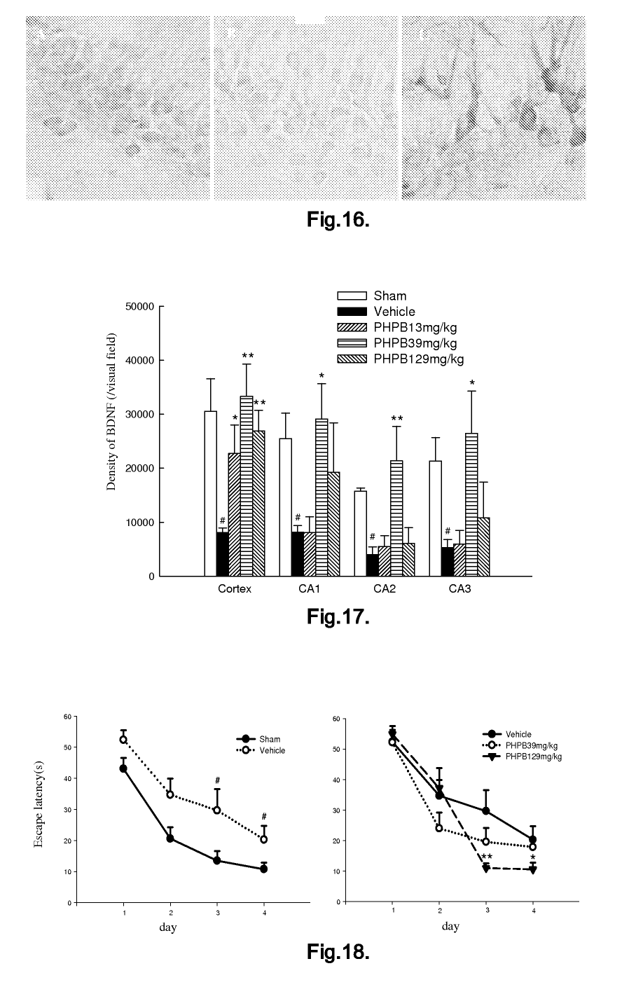

Brain-derived neurotrophic factor (BDNF) can maintain the survival and development of neurons. It is generally present in normal brain tissue of animals, but reaches high expression in early ischemia and declines rapidly after 24 h to the general expression level. In the cortex and hippocampus, there are no significant differences in treatment groups and vehicle group based on the area of BDNF immunohistochemistry (data not shown). However, based on staining intensity, expression of BDNF was significantly reduced both in the cortex or hippocampus of 2-VO rats, compared with sham group. In the cortex, dl-PHPB could increase the expression of BDNF, strongestly at the dose of 39 mg/kg, succeeded by 129 mg/kg and 13 mg/kg (FIGS. 13 and 17). In the hippocampus CA1, CA2, CA3 area, dl-PHPB 39 mg/kg significantly increased BDNF expression (P<0.05 or P<0.01), dl-PHPB 129 mg/kg increased BDNF is only a trend without statistical significance (FIG. 14-17). As the dye is proportional to density and BDNF levels, and the staining area within a certain range does not take into account the depth of staining, the staining density is more accurately than the stained area to reflect the content of BDNF. These results suggest that, dl-PHPB could increase the level of BDNF in brain tissue of 2-VO rats.

### Example 2

### The Study of Aβ(25-35)-Induced Dementia in Rat: dl-PHPB Improves the Memory and Spatial Learning Abilities of Dementia Rats

### 1. Materials And Methods

dl-PHPB was offered by the department of medical synthetic chemistry of our institute. dl-PHPB was dissolved in PBS. Aβ(25-35) was purchased from SIGMA. The levels of SOD, ChAT, and MDA activities were determined with commercial colorimetric assay kits (Nanjing Jiancheng Bioengineering Institute, China). The content of protein in the supernatant was determined by Bradford method using BSA as the standard.

### 2. Instrument

The water maze apparatus was designed by Institute of Material Medica, Chinese Academy of Medical Sciences. Enzyme mark instrument (MQX 200) was purchased by Bio Tek Instruments. Paraffin section machine (IR2135) purchased by German Leica co., LTD. Thermostatic freezing section machine (620-E) purchased by UK Shandon co., LTD. Automatic microphotography system (Nikon ECLIPSE 80i) purchased by Japanese Nikon Corporation.

### 3. Generation of Aβ(25-35)-Induced Dementia in Rat

Aβ(25-35)-induced dementia model in 10 months of Male Wistar rats with 600 g B.W. was created by slow injection of 15 nmol (volume of 50) aggregation of Aβ (25-35). Sham-operation group was only injected with PBS (volume of 50). After operation, the animals were injected penicillin 200 000 units for 4 days by intraperitoneal injection.

### 4. Treatment Groups and Drug Administration.

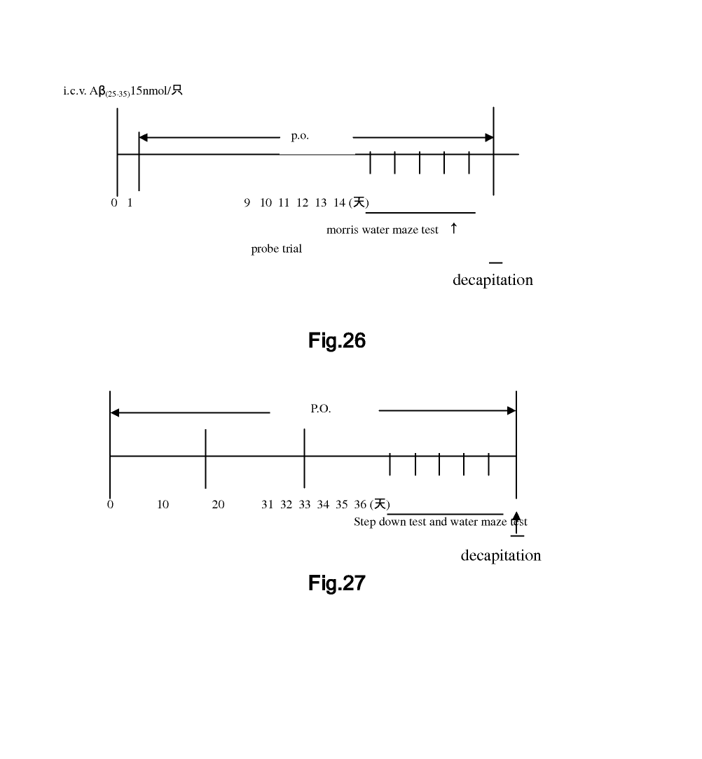

Rats were divided into four experimental groups randomly (10 rats/each group): one sham-operated group, one vehicle control group, and two dl-PHPB-treated groups (39, and 129 mg/kg). At 1 days after operation, drugs and vehicle were administered orally to rats for 14 days (one time/each day). The spatial learning and memory abilities of rats were detected by Morris water maze in the 9-12 days after operation. The testing of Morris water was commenced at 40 min after drugs treated. The biochemical and pathology assays conducted later (in 24 h) after behavioral testing. The experimental schedule is shown in FIG. 26.

### 5. Morris Water Maze

The navigation test was performed in 9-12 days after icy Aβ (25-35) by the same method as the before. On the thirteenth day, the platform was removed and the rat was allowed to search for the platform for 30 s (probe test). The time in the platform quadrant and latency time to cross platform location were recorded to measure the spatial learning ability without the influence of chance encounters with the platform.

### 6. Biochemical Assays

The methods as described previously.

### 7. Statistical Analysis

The results were expressed as mean±SEM. The data from training trail in the Morris water maze were analyzed by two way analysis of variance (ANOVA) to detect the difference between groups and over time. The post hoc Dunnett's test was used to test the differences between two groups. Probe trial, biochemical assay, and immunohistochemistry assay were statically analyzed using one-way ANOVA followed by post hoc Dunnett's test. The results were considered to show a significant difference when the p value was less than 0.05.

### Results

1. dl-PHPB Significantly Improved Aβ (25-35)-Induced Impairments in Spatial Learning and Spatial Working Memory of Rats.

In the navigation test, the rats were trained for 4 days. The latency of each group gradually reduced, indicating the memory capacity for the location of the security platform in each group was growing.

In the 1^{st} day, there was no significant difference in each group. But the escape latency of vehicle group was longer than sham group. In the 2^{nd} day, the escape latency of dl-PHPB group (39 mg/kg) was shorter than sham group (P<0.05). From 3^{rd} to 4^{th} days, the escape latency of vehicle group was significantly longer than sham group (P<0.05). However, the escape latency of dl-PHPB group (129 mg/kg) was significantly shorter than vehicle group (P<0.05 or P<0.01). The escape latency of dl-PHPB group (39 mg/kg) was shorter than vehicle group. The results shown that dl-PHPB could reduce the escape latency of Aβ (25-35)-induced dementia in dose-dependent manner. Namely, dl-PHPB could improve learning and memory abilities of Aβ (25-35)-induced dementia rats (FIG. 18). After 4 days' training, the swimming speed of rats in each group was no significantly different (data not shown), indicating the icy Aβ (25-35) in rats does not affect the physical status. Water maze test can reliably reflect the ability of learning and memory of animals.

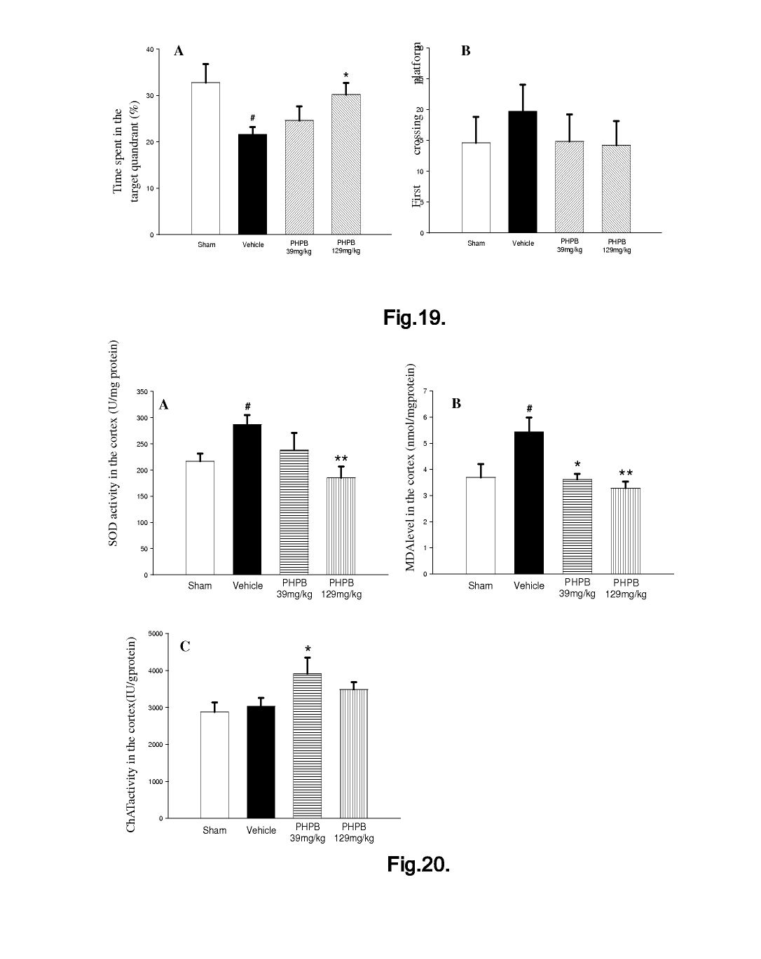

In probe test, animals of vehicle group has significantly the less percentage of time in the target quadrant (21.6±1.6%) than the sham group (32.8±4.0%, P<0.05), and dl-PHPB (129 mg/kg) could significantly increase the percentage of time in the target quadrant (30.2±2.5%) than vehicle group (p<0.05), dl-PHPB (39 mg/kg) only had the longer trend (24.6±3.0%) than vehicle group (FIG. 19A). dl-PHPB could increased the time percentage of the target quadrant in dose dependent manner. The animals of vehicle group had the longer trend than sham group in the time of first cross the platform location. Compared with the sham group, dl-PHPB also shorten trend in the time of the first cross platform. If the number of animal in each group was increased, the time of the first cross platform may be appearing significant difference (FIG. 19B).

In conclusion, dl-PHPB could improve the short memory and spatial learning impairment of Aβ (25-35)-induced dementia of rats in dose-dependent manner.

2. Effects of dl-PHPB on the SOD, ChAT Activity and MDA Level of Brain Tissues from Dementia Rats.

SOD plays an important role in maintenance of oxygen free radicals balance, can effectively eliminate oxygen free radicals and reduce oxidative damage. MDA is one of the main peroxides. SOD activity may reflect the antioxidant levels in brain tissue, while the level of MDA in brain tissues reflecting the situation of lipid peroxidation. The results showed that the SOD activity of Aβ (25-35)-induced dementia rats significantly increased by 32% (286.8±18.3 U/mg protein), compared with sham operation (216.9±14.5 U/mg protein). After oral administration dl-PHPB 39 mg/kg and 129 mg/kg for 2 weeks, the SOD activities in the cortex were decreased to 238.2±32.7 and 185.2±21.6 U/mg protein, the later had significant difference (P<0.01), compared with vehicle group. dl-PHPB could decrease the cortical SOD activity in dose dependent manner (FIG. 20A). In the hippocampus, the SOD activity of Aβ (25-35)-induced dementia rats did not significant differ, dl-PHPB has not significantly improved in SOD activity.

In the cortex, the MDA level of vehicle group (5.43±0.55 nmol/mg protein) was significantly increased, compared with sham operation group (3.69±0.52 nmol/mg protein) (P<0.05). After oral administration dl-PHPB 39 mg/kg and 129 mg/kg for 2 weeks, the cortex MDA levels significantly decreased to 3.62±0.21 and 3.28±0.25 nmol/mg protein, compared with the sham operation group (P<0.05 and P<0.01). The results shown that dl-PHPB could decrease the cortical MDA level of Aβ (25-35)-induced dementia rats in dose dependent manner (FIG. 20B).

Acetylcholine is a neurotransmitter in the central nervous system, mediated by cholinergic nerve signaling. Acetylcholine is closely related with learning and memory, and synthesized by ChAT. So, the activity of ChAT can indirectly reflect levels of acetylcholine and the status of cholinergic function. In rats with Aβ (25-35)-induced dementia, the cortical ChAT activity did not change significantly, compared with sham operation group. After administration of 2 weeks, 39 mg/kg of dl-PHPB significantly increased the ChAT activity (P<0.05), 129 mg/kg is also a strong trend. The results shown that the dl-PHPB may improve the ChAT activity in Aβ (25-35)-induced dementia rats (FIG. 20C).

In conclusion, dl-PHPB could decrease the SOD activity and MDA level of Aβ (25-35)-induced dementia rat in a dose dependent manner. dl-PHPB reduces lipid peroxidation and restores normal brain tissue oxidation and antioxidant homeostasis. In addition, dl-PHPB may increase ChAT activity and improve the cholinergic function in cortex of Aβ (25-35)-induced dementia rats. However, the effect of dl-PHPB on the cortical ChAT activity in normal rats requires further studies.

### Example 3

### The Study in SAMP8 Mice: dl-PHPB Improves the Memory and Spatial Learning Abilities of SAMP8 Mice

### 1. Materials and Methods

dl-PHPB was offered by the department of medical synthetic chemistry of our institute. dl-PHPB was dissolved in PBS. The levels of MDA, SOD, ChAT and ATPase activities were determined with commercial colorimetric assay kits (Nanjing Jiancheng Bioengineering Institute, China). The content of protein in the supernatant was determined by Bradford method using BSA as standard.

### 2. Instrument

The water maze apparatus and DTT-2 jumping apparatus were designed by Institute of Material Medica, Chinese Academy of Medical Sciences. Enzyme mark instrument (MQX 200) was purchased from Bio Tek Instruments. Paraffin section machine (IR2135) purchased from German Leica co., LTD. Thermostatic freezing section machine (620-E) purchased from UK Shandon co., LTD. Automatic microphotography system (Nikon ECLIPSE 80i) purchased from Japanese Nikon Corporation.

### 3. Animals

SAMP8 in 10 months old, male, SPF grade, purchased from Beijing Vital River Laboratory Animal Technology Co. Ltd.

### 4. Treatment Groups and Drug Administration

SAMP8 were divided into three experimental groups randomly: one vehicle control group, and two dl-PHPB-treated groups (50, and 160 mg/kg). Drugs and vehicle were administered orally to SAMP8 mice for 35 days (one time/each day). The spatial learning and memory abilities of SAMP8 mice were detected by Morris water maze and DTT-2 jumping apparatus in 31-35 days. The testing was commenced at 40 min after drugs treated. The biochemical and pathology assays conducted later (in 24 h) after behavioral testing. The experimental schedule is shown in FIG. 27.

### 5. Step Down Test

The step-down avoidance task was performed by using a behavior box (22 cm×15 cm×30 cm each). Each testing chamber has 3 Plexiglas black walls, a clear Plexiglas front wall, metal grid floors and an insulating platform (3 cm diameter, 4 cm height), which is located at one corner of the testing chamber. The metal grids are connected to the output terminals of an electrical stimulator. For the habituation session, each mouse was gently placed on the insulating platform and was allowed to explore in the testing chamber for 3 min before placing back on the platform again. Monophasic pulses (1 ms, 1 Hz, 36 VDC) were continuously delivered for 5 min during the training tria. If the mouse steps down from the platform onto the grid floor, the mouse will be subjected to receive an electric shock until returning to the platform. Then, 24 h after the training, the mice were placed on the platform for assessing their long term memory of retention period. The electric shocks were delivered for 5 min and the latency to step down on the grid with four paws for the first time (step-down latency) and the numbers of errors subjected to shocks within 5 min were recorded.

### 6. Water Maze Test

The water maze consisted of square black opaque plastic box (80 cm×50 cm×20 cm), of which four blind-side and a terminal stage. When a black plastic sheet (15 cm×20 cm) was placed in different locations, there were different the starting points and different number of blind side. The water maze filled with water at 25±1° C. to a depth 12 cm. Mice can be placed in different starting point, making experience with different number of blind side to the end. The number of errors into the blind side and the time to reach the end platform (escape latency) was recorded.

Each mouse was permitted to stay there for 5 s and place in the pool, allowed 3 min to find the end platform. The escape latency (time to reach the platform) was used to assess acquisition of the water maze task. Sessions was repeated for 4 consecutive days.

### 7. Biochemical Assays

The methods as described previously.

### 8. Statistical Analysis

The results were expressed as mean±SEM. The data from training trail in the Morris water maze were analyzed by two way analysis of variance (ANOVA) to detect the difference between groups and over time. The post hoc Dunnett's test was used to test the differences between two groups. Probe trial, biochemical assay, and immunohistochemistry assay were statically analyzed using one-way ANOVA followed by post hoc Dunnett's test. The results were considered to show a significant difference when the p value was less than 0.05.

### Results

1. dl-PHPB Significantly Rescued Impairments in Spatial Learning and Spatial Working Memory of SAMP8 Mice.

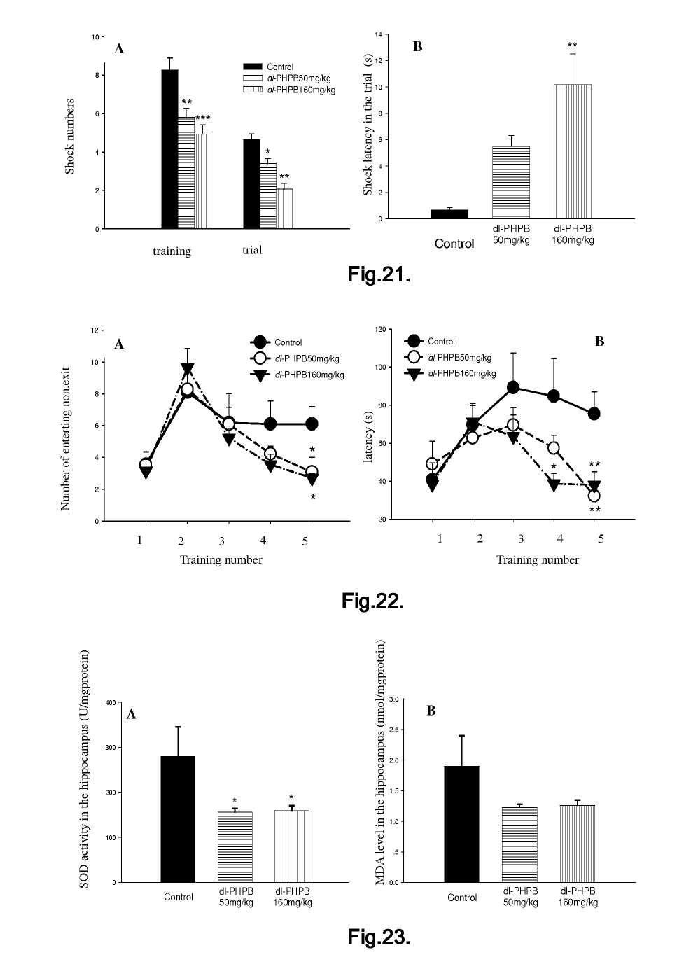

Step down test is a typical experiment to detect the ability of avoidance respond of animals. The avoidance response capacity of animals is measured based on the first time (step-down latency) and the numbers of errors subjected to shocks. In the first day of training, dl-PHPB (50 and 160 mg/kg) significantly decreased the numbers of errors subjected to shocks (5.8±0.5, 4.9±0.5 Vs 8.3±0.6) in a dose dependent manner (P<0.01 and P<0.001). In the second day, the dl-PHPB also significantly decreased the numbers of errors subjected to shocks (3.4±0.3, 2.1±0.3), compared with vehicle group (4.6±0.3) (P<0.05 and P<0.01). And dl-PHPB could significantly increased step-down latency time (5.5±0.8, 10.2±2.4) in a dose dependent manner, compared with vehicle group (0.7±0.2) (P<0.01) (FIG. 21), The experiment results indicated dl-PHPB (50 and 160 mg/kg) could enhance the ability of active and passive avoidance response, and improve the ability of learning and memory.

Water maze test is commonly used to detect the ability of recent memory and spatial learning memory in mice. The ability of learning and memory is evaluated base on the number of errors into the blind side and the time to reach the end platform (escape latency).

In this study, the first and second training and testing sessions include 2 and 3 blind-side, respectively. However, there are four blind-side from third to fifth training and testing sessions. During the first three trainings, there was no significant difference in the number of errors into the blind-side of SAMP8, compared with the vehicle group. In the later three trainings, dl-PHPB decreased the number of errors into the blind-side of SAMP8, compared with the vehicle group. dl-PHPB significantly decreased the number of errors into the blind-side of SAMP8 in the fourth training, compared with the vehicle group. In the fifth test, the number of errors into the blind-side in SAMP8 treated by dl-PHPB (respectively 3.1±0.9 and 2.7±0.3) was significantly less than in the vehicle group (6.1±1.1) (P<0.05).

In the first and second trainings, there was no significant difference on the escape latency time of each group. In the third training, dl-PHPB treated groups displayed the tendency of the shorter escape latency. However, dl-PHPB at 160 mg/kg treatment significantly shortened the escape latency time in the fourth training (P<0.05). In the last test, dl-PHPB at 50 and 160 mg/kg treatment group could significantly shorten the escape latency time (P<0.01), compared with vehicle group (FIG. 22). Those, dl-PHPB can improve spatial learning and memory defects of SAMP8 mice.

In summation, dl-PHPB at 50 and 160 mg/kg doses could significantly improve short-term memory and spatial learning abilities of SAMP8 mice in a dose dependent manner.

2. Effects of dl-PHPB on the SOD Activity and MDA Level of Brain Tissues from SAMP8 Mice.

SOD plays an important role in maintenance of oxygen free radicals balance, and can effectively eliminate oxygen free radicals and reduce oxidative damage. MDA is one of the main peroxides. SOD activity may reflect the antioxidant levels in brain tissue, while the level of MDA in brain tissues reflects the situation of lipid peroxidation. The results showed that the SOD activity of SAMP8 mice was 279.4±65.7 U/mg protein in the hippocampus. After oral administration of dl-PHPB 50 mg/kg and 160 mg/kg for 35 days, the SOD activities in the hippocampus were decreased to 156.2±7.8 and 158.7±11.4 U/mg protein, which both had significant differences (P<0.05), compared with vehicle group. dl-PHPB could decrease the SOD activity of hippocampus in a dose dependent manner (FIG. 23A). In the cortex, the SOD activity after dl-PHPB treatment for 35 days did not significantly improve SOD activity.

After oral administration dl-PHPB 50 mg/kg and 160 mg/kg for 35 days, the MDA levels in the hippocampus were 1.23±0.05 and 1.26±0.09 nmol/mg protein, decreased by 35.3% and 33.7%, compared with SAMP8, but without statistical significance (FIG. 23B). In the cortex, the MDA level after dl-PHPB administration for 35 days did not significantly improved in MDA level (data not shown).

The results indicated that dl-PHPB could alleviate the disorders of the brain anti-oxidation, decrease production of lipid peroxidation, and restore the balance of oxidation.

3. Effects of dl-PHPB on the ChAT and AChE Activity of Brain Tissues, and ATP Level of Mitochondria in the Cortex of SAMP8 Mice.

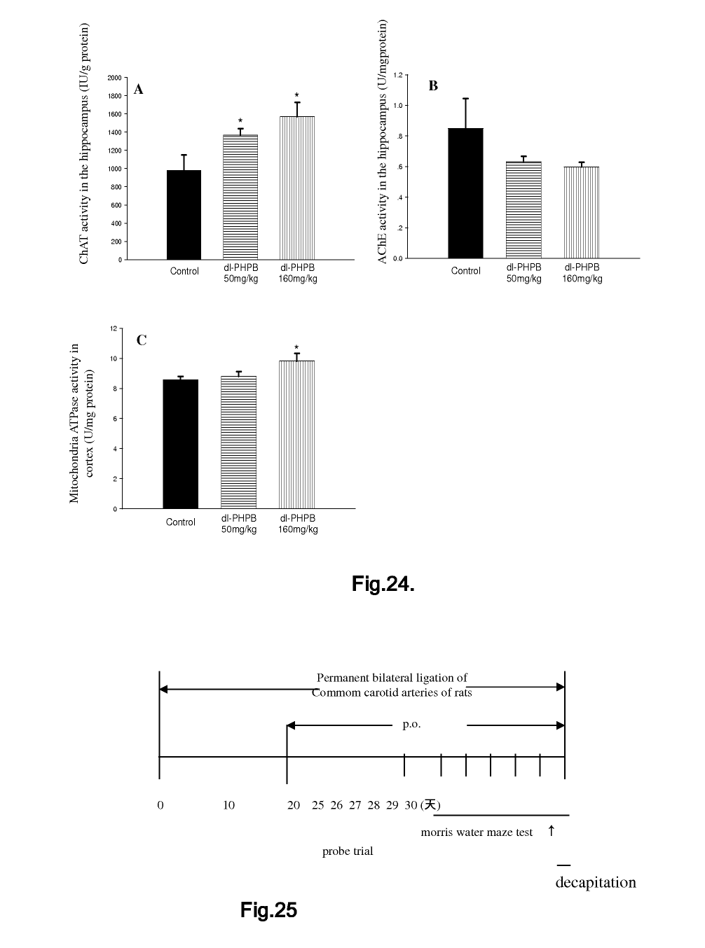

Acetylcholine is a neurotransmitter in the central nervous system, mediated by cholinergic nerve signaling. Acetylcholine is closely related with learning and memory, and synthesis by ChAT. So, the activity of ChAT can indirectly reflect levels of acetylcholine and the status of cholinergic function. After administration of dl-PHPB for 35 days, dl-PHPB significantly increased ChAT activity (P<0.05) in the hippocampus in a dose-dependent manner (FIG. 24A). In the cortex, dl-PHPB did not significantly improve ChAT activity (data not shown). The results showed dl-PHPB may improve the ChAT activity of SAMP8 (FIG. 24B).

Mitochondrial ATPase plays a key role in the mitochondrial function. After oral administration of dl-PHPB at the doses of 50 mg/kg and 160 mg/kg for 35 days, only 160 mg/kg dose significantly increased the ATPase activity (9.82±0.51 U/mg protein), compared with vehicle group (8.58±0.21 U/mg protein) (FIG. 24C).

In conclusion, dl-PHPB can dose-dependently increase the ChAT activity of hippocampus in SAMP8 mice. The results implied that PHPB might ameliorate the cholinergic function via increasing the ACh content in the hippocampus of SAMP8 mice.

1. A method for the prevention, amelioration and treatment of dementia or its symptoms, comprising administering to a subject in need a therapeutically effective amount of potassium 2-(1-hydroxypentyl)-benzoate (PHPB).

2. The method of claim 1, wherein features of dementia include Alzheimer's, vascular dementia or the mixed of both types.

3. The method of claim 2, wherein features in Alzheimer's disease or symptom include the memory loss, cognitive dysfunction, slow thinking or spatial disorientation.

4. A method for reduction of insults induced by oxygen stress in the brain, enhancement of functions in the cholinergic nerve, protection of neurons, or increase of brain-derived growth factor, comprising administering to a subject in need an effective amount of potassium 2-(1-hydroxypentyl)-benzoate.

5. The method of claim 4, wherein oxidative stress in brain injury is reduced by inhibiting the abnormal brain compensatory increase of antioxidant enzyme activity, and lipid peroxidation is reduced, and normal brain tissue oxidation-anti oxidation homeostasis is restored.

6. The method of claim 5, wherein the antioxidant enzymes are superoxide dismutase, lipid peroxidation is MDA.

7. The method of claim 4, wherein improvement of cholinergic function is achieved by increasing ChAT activity and inhibiting AChE activity.

8. A pharmaceutical composition for the prevention, alleviation and treatment on the dementia-related signs, comprising a therapeutically effective amount of PHPB, and one of pharmaceutical acceptable carriers and excipients.

9. The composition of claim 8, wherein features of dementia include Alzheimer's, vascular dementia or the mixed of both types.

10. The composition of claim 8, wherein features in Alzheimer's disease or symptom include the memory loss, cognitive dysfunction, slow thinking or spatial disorientation.

11. The composition of claim 8, prepared as one of the following formulations on the basis of administration route: solution, suspension, emulsion, pill, capsule, powder, controlled-release or sustained-release preparation.

12. A method for the prevention, alleviation and treatment on the dementia-related signs, comprising administering to a subject in need, a therapeutically effective amount of the pharmaceutical composition of claim 8.

13. The method of claim 12, wherein features of dementia include Alzheimer's, vascular dementia or the mixed of both types.

14. The method of claim 13, wherein features in Alzheimer's disease or symptom include the memory loss, cognitive dysfunction, slow thinking or spatial disorientation.

15. The method of claim 12, wherein the administration route is selected from the group consisting of parenteral, per os, focal, intracutaneous, intramusculary, intraperitoneally, subcutaneous, and intranasal route.

16. The method of claim 12, wherein said therapeutically effective amount of PHPB is from 0.5 to 200 mg/kg body weight.

17. The method of claim 12, wherein said therapeutically effective amount of PHPB is 2-100 mg/kg body weight.

18. The method of claim 12, wherein said therapeutically effective amount of PHPB is 3-50 mg/kg body weight.

19. The method of claim 12, wherein said therapeutically effective amount of PHPB is 4-35 mg/kg body weight.

20. The method of claim 12, wherein said therapeutically effective amount of PHPB is 5-20 mg/kg body weight.
